(19) United States
(12) Patent Application Publication
(10) Pub. No.: US 2009/0075827 A1
(43) Pub. Date: Mar. 19, 2009

(54) DIAGNOSTIC METHODS AND AGENTS

(75) Inventors: Ross Young, Lutwyche (AU); Bruce Lawford, Mt. Gravatt East (AU); C. Phillip Morris, Indooroopilly (AU); Joanne Voisey, Kedron (AU); Ian Hughes, Corinda (AU); Christopher Dean Swagell, Southport (AU)

Correspondence Address: KNOBBE MARTENS OLSON & BEAR LLP, 2040 MAIN STREET, FOURTEENTH FLOOR, IRVINE, CA, 92614, US

(73) Assignee: QUEENSLAND UNIVERSITY OF TECHNOLOGY, Brisbane (AU)

(21) Appl. No.: 12/016,438

(22) Filed: Jan. 18, 2008

Related U.S. Application Data
(60) Provisional application No. 60/885,837, filed on Jan. 19, 2007.

(51) Int. Cl. C40B 30/00; C40B 40/06; C12Q 1/68; C12Q 1/02; G01N 33/566

(52) U.S. Cl. 506/7; 506/16; 435/6; 435/29; 435/7.21

(57) ABSTRACT

The present invention relates generally to a method and agents for profiling or stratifying an individual or group of individuals with respect to a neurological, psychiatric or psychological condition, phenotype or state, including a sub-threshold neurological, psychiatric or psychological condition, phenotype or state. More particularly, the present invention utilizes genetic means to profile or stratify individuals with respect to a neurological, psychiatric or psychological condition, phenotype or state. The present invention enables the identification of individuals at risk of these disorders thus affording the opportunity for early intervention. In addition, the subject invention allows the prediction of drug or other treatment response and adverse reactions.

### RELATED APPLICATIONS

This application claims the benefit of U.S. Provisional Application No. 60/885,837 filed Jan. 19, 2007, which is hereby expressly incorporated by reference in its entirety.

### FIELD OF THE INVENTION

The present invention relates generally to a method and agents for profiling or stratifying an individual or group of individuals with respect to a neurological, psychiatric or psychological condition, phenotype or state, including a sub-threshold neurological, psychiatric or psychological condition, phenotype or state. More particularly, the present invention utilizes genetic means to profile or stratify individuals with respect to a neurological, psychiatric or psychological condition, phenotype or state. The present invention enables the identification of individuals at risk of these disorders thus affording the opportunity for early intervention. In addition, the subject invention allows the prediction of drug or other treatment response and adverse reactions.

### DESCRIPTION OF THE RELATED ART

Reference to any prior art in this specification is not, and should not be taken as, an acknowledgment or any form of suggestion that this prior art forms a part of the common general knowledge in any country.

Psychological “disorders” are endemic in many societies. Reference to “disorders” in this context means that an individual exhibits behavioral patterns which are inconsistent with societal norms. Most psychological phenotypes have both environmental and genetic risk factors. The basis of these disorders is in many cases significantly genetic. For example, early detection of disorders such as schizophrenia using genetic technology has considerable potential to identify those at risk prior to the development of this chronic condition. Commencement of a low dose antipsychotic regime and early cognitive behavioral therapy may prevent the emergence of the full disorder. Development of the full disorder is associated with significant impairment of social, cognitive and occupational functioning. In Australia, the morbidity and mortality data for those with schizophrenia are similar to the poor health outcomes faced by other disenfranchised groups such as Indigenous Australians.

Schizophrenia is a particularly complex psychological phenotype. Schizophrenia is a common, chronic, disabling illness with an incidence of 15 new cases per 100,000 population per year (Kelly et al, Ir. J. Med. Sci. 172:37-40, 2003). Additionally, “unaffected” first degree relatives show both child (Niendam et al, Am. J. Psychiatry. 160:2060-2062, 2003) and adult (MacDonald et al, Arch. Gen. Psychiatry. 60:57-65, 2003) deficits in cognitive functioning. Siblings of those with schizophrenia also exhibit an abnormal MRI response in the dorsolateral prefrontal cortex implicating inefficient information processing (Callicott et al, Am. J. Psychiatry. 160:709-719, 2003). Furthermore, both those with schizophrenia and their unaffected siblings show both reductions in hippocampal volume and hippocampal shape deformity (Tepest et al, Biol, Psychiatry. 54:1234-1240, 2003). Decreased temporoparietal P300 amplitude and increased frontal P300 amplitude are found in both schizophrenic patients and their siblings (Winterer et al, Arch. Gen. Psychiatry. 60:1158-1167, 2003). Taken together, these findings indicate that the underlying pathophysiological state of schizophrenia is considerably more widespread in the general population than prevalence figures for schizophrenia would suggest and that a considerable genetic vulnerability for this disorder exists.

The apparent high genetic risk for schizophrenia has led to considerable research efforts aimed at the identification of susceptibility genes. This has resulted in linkages or associations with regions 6p21-22, 1q21-22 and 13q32-34 with single studies reporting significance at P<0.05 (Owen et al, Mol. Psychiatry. 9:14-27, 2004) although a large multi-centre linkage study of schizophrenia loci on chromosome 22q failed to find any evidence for linkage or association to schizophrenia (Mowry et al, Mol. Psychiatry. 2004). Other regions that may be implicated include 8p21-22, 6q21-25, 5q21-q33, 10p15-p11 and 1q42 (Owen et al, supra 2004). Despite this limited progress, the conclusive identification of specific molecular genetic etiological factors in the pathogenesis of schizophrenia has not occurred (Miyamoto et al, Mol. Intervent. 3:27-39, 2004). However, it is clear from epidemiological studies (Gottesman I I et al, Proc. Natl. Acad. Sci. USA. 58:199, 1967; McGue M et al, Arch Gen Psych. 46:478-480) that the mode of transmission is complex and the predisposition of disease is most likely due to multiple genes exerting modest to small effect (Risch, Gen Epidemiol, 7:3-16, 1990).

Several lines of evidence have implicated the dopamine 2 receptor (DRD2) gene and genes related to neurotransmitters that influence dopamine function as candidates gene for schizophrenia genetic susceptibility. For example, all anti-psychotic medications are either antagonists or partial agonists of DRD2. DRD2 receptor has been repeatedly demonstrated to be the primary site of action for these medications (Seeman and Kapur, Proc. Natl. Acad. Sci. USA 97:7673-7675, 2000) indicating that schizophrenic symptoms are ameliorated by a reduction in DRD2 function. Additionally, recent evidence strongly suggests that schizophrenic patients have increased brain DRD2 density (Abi-Dargham et al, Proc. Natl. Acad. Sci. 97:8104-8109, 2000). Recently, several genotyping studies have identified an association of the 957C>T polymorphism (rs6277) of the DRD2 gene and schizophrenia in Northern European, Spanish and Finnish populations (Lawford et al, Schizo Res, 73:31-37, 2005; Hoenicka et al, Acta Psychiatr Scand, 114:435-438, 2006; Hanninen et al, Neuros Lett, 407:195-198, 2006). In addition, a gene located in close proximity to the DRD2 locus, the ankyrin repeat and protein kinase domain-containing protein 1 (ANKK1) gene, has been associated with schizophrenia (Dubretret et al, Schizophrenia Res, 49:202-212, 2001; Dubretret et al, Schizophrenia Res, 67:75-85, 2004).

Dystrobrevin binding protein 1 or dysbindin (DTNBP1) is the most convincing susceptibility gene for schizophrenia to date (Norton et al, Curr Opin Psychiatr, 19:158-164, 2006). DTNBP1 maps to chromosome 6p22.3 which is a consistently replicated schizophrenia linkage region (Group, Am J Med Genet, 67:580-594, 1996; Lewis et al, Am J Hum Genet, 73:34-48, 2003). Screening of exons has failed to identify mutations that associate non-synonymous alleles with disease, suggesting that susceptibility variants may affect mRNA expression or processing (Williams et al, Arch Gen Psychiatry, 61:336-344, 2004). Recent studies have shown that schizophrenia patients have lower mRNA concentrations in the prefrontal cortex and midbrain when compared to controls (Talbot et al, J Clin Invest, 113:1353-1363, 2004; Weickert et al, Arch Gen Psychiatry, 61:544-555, 2004). The mechanisms of vulnerability to schizophrenia remain uncertain, however, and recent study has shown DTNBP1 to be located presynaptically in glutamatergic neurons (Talbot et al, supra 2004). This observation may confer the vulnerability of schizophrenia through aberrant regulation of glutamatergic neurotransmission.

Regulator of G-protein signalling 4 (RSG4) is located within a putative linkage region at chromosome 1q21-22. A global study of expression has identified a decrease in RSG4 expression in prefrontal cortex of individuals with schizophrenia (Mimics et al, Mol Psychiatry, 6:293-301, 2001; Chowdari et al, Hum Mol Genet, 11(12):1373-1380, 2002). In addition variants in the 5′ flanking region and the first intron are modestly associated with increased schizophrenia vulnerability (Owen et al, Psychiatric genetics and genomics, 247-266, 2002). The mechanism of susceptibility to schizophrenia remains unclear. However, the RSG4 gene product down-regulates effects at G-protein-coupled receptor including the dopamine and serotonin receptors. Furthermore, RSG4 expression is modulated by stress (Ni et al, J Neurosci, 19(10):3674-3680, 1999) and is a known contributor factor to major metal illness, including bi-polar disorder (Berrettini, Am J Med Genet, 123C:59-64, 2003; Shifman et al, Am J Hum Genet, 71:1296-1303, 2002). Therefore, it is possible that RSG4 plays a role through these pathways to confer susceptibility to schizophrenia.

Catechol-O-methyl transferase (COMT) is localized to chromosomal region 22q11 and has been identified as the second greatest risk factor for mental illness (Bassett et al, Biol Psychiatry, 46:882-891, 1999; Murphy K C, Lancet, 359:426-430, 2002). COMT catalyzes the O-methylation of catecholamine neurotransmitters and catechol hormones leading to their inactivation. In addition, COMT also shortens the biological half-lives of certain neuroactive drugs, like L-DOPA, alpha-methyl DOPA and isoproterenol. Reports implicating the role of COMT in schizophrenia have been inconsistent, however, the weight of evidence seems to favour the involvement of COMT in the pathogenesis of schizophrenia (Shifman et al, supra 2002). A functional variant has been demonstrated to play a role in regulating aspects of cognitive functioning (Egan et al, Proc. Natl. Acad. Sci., USA, 98(12):6917-6922, 2001) and it has been proposed that decreased expression of COMT is involved in schizophrenia (Bray et al, Am J Hum Genet, 73(J):152-161, 2003). In addition, COMT has been implicated in schizophrenia and bi-polar disorder implying that these conditions share some genetic vulnerability factors (Berrettini, supra 2003; Shifman et al, supra 2002). However, the pathogenic mechanisms of COMT are yet to be fully elucidated. Imbalances in glutamate have been implicated in the pathogenesis of a number of psychiatric disorders including schizophrenia. The metabotropic glutamate receptor 3 (GRM3) is a receptor for glutamate and has been mapped to chromosome 7q21.1 (Scherer et al, Genomics, 31:230-233, 1996) and may contribute to a genetic predisposition to schizophrenia (Marti et al, Am J Med Genet, 144:46-50, 2002; Fujii Y et al, Psychiatr Genet, 13:71-76, 2003).

Genetic analysis has implicated the disrupted in schizophrenia 1 (DISC1) gene with a spectrum of major mental illness including schizophrenia, schizoaffective disorder, bipolar affective disorder and major depression. Emerging evidence from these studies have identified a casual relationship between DISC1 and directly measurable traits such as working memory, cognitive aging decreased gray matter volume in the prefrontal cortex, abnormalities in hippocampal structure and function and a reduction in the amplitude of the P300 event-related potential. DISC1 binds to a number of proteins involved in essential functions of neuronal function including neural migration, neurite outgrowth, cytoskeletal modulation and signal transduction (Yamada et al, Biol Psychiatry, 56:683-690, 2004; Millar et al, Science, 310:1187-1191, 2005).

5-Hydroxytryptamine (serotonin) receptor 2A (HTR2A) has been implicated as a functional candidate in many neuropsychiatric phenotypes including: schizophrenia, attention deficit hyperactivity disorder (ADHD), affective disorders, eating disorders, anxiety disorders, obsessive-compulsive disorder, suicide and Alzheimer's disease (AD) (Norton et al, Ann Med, 37(2):121-129, 2005). HTR2A is one of the several different receptors for 5-hydroxytryptamine (serotonin), a biogenic hormone that functions as a neurotransmitter, a hormone, and a mitogen. The HTR2A receptor mediates its action by association with G proteins that activate a phosphatidylinositol-calcium second messenger system.

A number of other genes have been associated with an increased susceptibility to schizophrenia. For example, associations between SNPs, haplotypes and mis-sense mutations have been associated with schizophrenia with the proline dehydrogenase (PRODH) gene (Liu et al, Proc. Natl. Acad. USA, 99:3717-3722, 2002). In addition, mice with an inactive PRODH gene have abnormalities of sensorimotor gating similar to those in humans that some consider a trait marker for schizophrenia (Gogos et al, Nat Genet, 21:434-439). A recent study of the karyopherin alpha 3 (KPNA3) gene, which functions in nuclear protein import as an adapter protein for nuclear receptor KPNB1, has implicated that it may contribute genetically to schizophrenia in a mall effect size (Wei et al, Neurosci Res, 52:342-346, 2005).

The present invention now provides a profile of risk factors and therapeutic targets useful in the diagnosis, treatment, monitoring and therapeutic drug development of neurological, psychiatric and psychological conditions, phenotypes and states.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention identifies a genetic link between genes encoding enzymes and receptors in dopaminergic pathways including and those genes related to GABAergic, glutamatergic, and serotonergic function which are likely to influence dopaminergic pathways and a neurological, psychiatric or psychological condition, phenotype or state. In particular, a population of individuals having related pathopsychological symptoms and behavioral patterns are shown to exhibit a particular polymorphism with the genetic regions of these genes. Even more particularly, the present invention identifies a ranking or profile of markers prevalent in individuals with schizophrenia or related neurological, psychiatric or psychological conditions including anxiety disorders, such as post traumatic stress disorder (PTSD) and addictions including alcohol dependence, nicotine dependence, and opioid dependence. Reference to a condition related to schizophrenia includes a condition having similar symptoms, underlying genetic cause or association and/or treatment rationale. The ranking or profile of markers enables stratification of individuals and groups of individuals including related individuals with respect to a disease condition, risk of developing a disease condition or the likelihood that an individual would be responsive to a particular treatment regime. The markers themselves are also potential drug targets.

Accordingly, the present invention contemplates a method for identifying a genetic profile associated with a neurological, psychiatric or psychological condition, phenotype or state including a sub-threshold neurological, psychiatric or psychological condition, phenotype or state in an individual or a group of individuals, said method comprising screening individuals for a polymorphism including a mutation in a gene selected from the list in Table 2, including its 5′ and 3′ terminal regions, promoter, introns and exons which has a statistically significant linkage or association to symptoms or behavior characterizing the neurological, psychiatric or psychological condition, phenotype or state or sub-threshold forms thereof. Reference herein to “a gene” includes one or more or all genes listed in Table 2. There may be multiple polymorphisms in one or more genes or a single polymorphism in each or a few genes.

In one particular embodiment, the genetic profile comprises from about one to about 100 single nucleotide polymorphisms (SNPs) in one or more genes listed in Table 2. In another embodiment, the genetic profile is a distribution panel as outlined in Table 3 or an individual gene therein. In another embodiment, the genetic profile comprises from one to 100 single or multiple nucleotide mutations such as insertions, additions, substitutions and deletions as well as rearrangements or microsatellites. All such nucleotide modifications are referred to herein as a “polymorphism”. Table 4 provides allele-distribution of control and schizophrenia patients.

Neurological, psychiatric or psychological conditions, phenotypes and states include, but are not limited to, addiction, dementia, anxiety disorders, bipolar disorder, schizophrenia, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia.

The present invention is particularly exemplified herein with respect to schizophrenia, anxiety disorder (eg PTSD), alcohol dependence, nicotine dependence and opioid dependence. These conditions are associated with polymorphisms in the genetic loci listed in Table 2 including 5′ and 3′ terminal regions, promoter, introns and exons therein. However, the present invention extends to the use of polymorphisms to profile individuals with respect to a range of conditions such as those listed above. In a particular embodiment, the genetic profile comprises one or more polymorphisms in two or more genes listed in Table 3. Reference to “one or more” includes from about one to about 100.

In an even more particular embodiment, the genetic profile is the panel in Table 3.

The present invention enables clinicians to make a genetic-based diagnosis of a neurological, psychiatric or psychological condition, phenotype or state or a risk or likelihood that an individual will develop such a neurological, psychiatric or psychological condition, phenotype or state. The invention allows the targeted implementation of treatment or preventative interventions including medication and cognitive-behavioral therapy to reduce the adverse consequences of the neurological, psychiatric or psychological condition, phenotype or state.

In addition, the identification of polymorphisms including mutations in genes associated with a neurological, psychiatric or psychological condition, phenotype or state enables agents to be identified which mask the physiological impact or consequences of the genetic profile. Consequently, agents which modulate levels of expressions of these genes are proposed to be useful in the treatment of schizophrenia, substance dependence, affective disorder, anxiety disorder or other neurological, psychiatric or psychological conditions, phenotypes or states.

Still further, the present invention enables individuals in therapy to be monitored and/or their treatment tailored depending on the presence of particular polymorphisms. Hence, the instant invention extends to personalized medicine and pharmacogenomic analysis and screening.

Accordingly, the present invention contemplates a method a method for determining the likelihood of a subject responding favorably to a particular drug in the treatment of a neurological, psychiatric or psychological condition, phenotype or state said method comprising obtaining or extracting a DNA sample from cells of said individual and screening for or otherwise detecting the presence of from about one to about 100 polymorphisms in one or more genes listed in Table 2 including their 5′ or 3′ terminal region, promoter, intron or exons which with a statistical significant association with a particular neurological, psychiatric or psychological condition, phenotype or state wherein the presence of the polymorphism profile is indicative of the likelihood of the drug being effective.

The present invention further provides a method for identifying a genetic basis behind diagnosing or treating a neurological, psychiatric or psychological condition, phenotype or state in an individual, said method comprising obtaining a biological sample from said individual and detecting a protein encoded by a nucleotide sequence having from about one to about 100 polymorphisms in one or more genes listed in Table 2 or Table 3 including their 5′ or 3′ terminal region, promoter, intron or exons with a statistical significant association with a particular neurological, psychiatric or psychological condition, phenotype or state resulting in from about one to about 100 amino acid insertions, substitutions or deletions wherein the presence of an altered amino acid sequence is indicative of the presence of a polymorphism and the likelihood of a neurological, psychiatric or psychological condition, phenotype or psychological condition, phenotype or state.

As indicated above, reference to a “polymorphism” includes, in one embodiment, a SNP; in another embodiment, a multiple nucleotide polymorphism (MNP); and in yet another embodiment, any nucleotide mutation such as an insertion, addition, substitution or deletion as well as rearrangements or microsatellites.

Reference to genes associated with schizophrenia, PTSD, alcohol dependence, nicotine dependence, opioid dependence or other neurological, psychiatric or psychological conditions, phenotypes or states maybe referred to herein as targets, genetic loci, alleles or a panel of genes.

In a particular embodiment, Table 3 provides a panel of genes and polymorphisms ranked according to diagnostic significance. However, the present invention extends to one or more polymorphisms in one or more particularly two or more genes in Table 3.

Microarrays, gene arrays and other high throughput diagnostic assays also form part of the present invention together with diagnostic and therapeutic kits.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A summary of the sequence identifiers is provided in Table 1. A sequence listing is provided after the claims.

A summary of the sequence identifiers used herein are shown in Table 1.

TABLE 1
Sequence Identifiers

| Sequence Identifier | Sequence |
|---|---|
| SEQ ID NO: 1 | Primer for DRD2 |
| SEQ ID NO: 2 | Primer for DRD2 |
| SEQ ID NO: 3 | Primer for DTNBP1 |
| SEQ ID NO: 4 | Primer for DTNBP1 |
| SEQ ID NO: 5 | Primer for DTNBP1 |
| SEQ ID NO: 6 | Primer for DTNBP1 |
| SEQ ID NO: 7 | Primer for GABRA1 |
| SEQ ID NO: 8 | Primer for GABRA1 |
| SEQ ID NO: 9 | Primer for DAT |
| SEQ ID NO: 10 | Primer for DAT |
| SEQ ID NO: 11 | Primer for DAT |
| SEQ ID NO: 12 | Primer for DAT |
| SEQ ID NO: 13 | Primer for DAT |
| SEQ ID NO: 14 | Primer for DAT |
| SEQ ID NO: 15 | Primer for DAT |
| SEQ ID NO: 16 | Primer for DAT |
| SEQ ID NO: 17 | Primer for COMT |
| SEQ ID NO: 18 | Primer for COMT |
| SEQ ID NO: 19 | Primer for COMT |
| SEQ ID NO: 20 | Primer for COMT |
| SEQ ID NO: 21 | Primer for DAT |
| SEQ ID NO: 22 | Primer for DAT |
| SEQ ID NO: 23 | Primer for RGS4 |
| SEQ ID NO: 24 | Primer for RGS4 |
| SEQ ID NO: 25 | Primer for KPNA3 |
| SEQ ID NO: 26 | Primer for KPNA3 |
| SEQ ID NO: 27 | Primer for AKT1 |
| SEQ ID NO: 28 | Primer for AKT1 |
| SEQ ID NO: 29 | Primer for HTR2A |
| SEQ ID NO: 30 | Primer for HTR2A |
| SEQ ID NO: 31 | Primer for PRODH |
| SEQ ID NO: 32 | Primer for PRODH |
| SEQ ID NO: 33 | Primer for PRODH |
| SEQ ID NO: 34 | Primer for PRODH |
| SEQ ID NO: 35 | Primer for PRODH |
| SEQ ID NO: 36 | Primer for PRODH |
| SEQ ID NO: 37 | Primer for ANKK1 |
| SEQ ID NO: 38 | Primer for ANKK1 |
| SEQ ID NO: 39 | Primer for DISC1 |
| SEQ ID NO: 40 | Primer for DISC1 |
| SEQ ID NO: 41 | Primer for GRM3 |
| SEQ ID NO: 42 | Primer for GRM3 |
| SEQ ID NO: 43 | Primer for RGS4 |
| SEQ ID NO: 44 | Primer for RGS4 |
| SEQ ID NO: 45 | Primer for DRD2 |
| SEQ ID NO: 46 | Primer for DRF2 |
| SEQ ID NO: 47 | Primer for DRF2 |
| SEQ ID NO: 48 | Primer for DTNBP1 |
| SEQ ID NO: 49 | Primer for DTNBP1 |
| SEQ ID NO: 50 | Primer for DTNBP1 |
| SEQ ID NO: 51 | Primer for DTNBP1 |
| SEQ ID NO: 52 | Primer for DTNBP1 |
| SEQ ID NO: 53 | Primer for DTNBP1 |
| SEQ ID NO: 54 | Primer for GABRA1 |
| SEQ ID NO: 55 | Primer for GABRA1 |
| SEQ ID NO: 56 | Primer for GABRA1 |
| SEQ ID NO: 57 | Primer for DAT |
| SEQ ID NO: 58 | Primer for DAT |
| SEQ ID NO: 59 | Primer for DAT |
| SEQ ID NO: 60 | Primer for DAT |
| SEQ ID NO: 61 | Primer for DAT |
| SEQ ID NO: 62 | Primer for DAT |
| SEQ ID NO: 63 | Primer for DAT |
| SEQ ID NO: 64 | Primer for DAT |
| SEQ ID NO: 65 | Primer for DAT |
| SEQ ID NO: 66 | Primer for DAT |
| SEQ ID NO: 67 | Primer for DAT |
| SEQ ID NO: 68 | Primer for DAT |
| SEQ ID NO: 69 | Primer for COMT |
| SEQ ID NO: 70 | Primer for COMT |
| SEQ ID NO: 71 | Primer for COMT |
| SEQ ID NO: 72 | Primer for COMT |
| SEQ ID NO: 73 | Primer for COMT |
| SEQ ID NO: 74 | Primer for COMT |
| SEQ ID NO: 75 | Primer for DAT |
| SEQ ID NO: 76 | Primer for DAT |
| SEQ ID NO: 77 | Primer for DAT |
| SEQ ID NO: 78 | Primer for RGS4 |
| SEQ ID NO: 79 | Primer for RGS4 |
| SEQ ID NO: 80 | Primer for RGS4 |
| SEQ ID NO: 81 | Primer for KPNA3 |
| SEQ ID NO: 82 | Primer for KPNA3 |
| SEQ ID NO: 83 | Primer for KPNA3 |
| SEQ ID NO: 84 | Primer for AKT1 |
| SEQ ID NO: 85 | Primer for AKT1 |
| SEQ ID NO: 86 | Primer for AKT1 |
| SEQ ID NO: 87 | Primer for HTR2A |
| SEQ ID NO: 88 | Primer for HTR2A |
| SEQ ID NO: 89 | Primer for HTR2A |
| SEQ ID NO: 90 | Primer for PRODH |
| SEQ ID NO: 91 | Primer for PRODH |
| SEQ ID NO: 92 | Primer for PRODH |
| SEQ ID NO: 93 | Primer for PRODH |
| SEQ ID NO: 94 | Primer for PRODH |
| SEQ ID NO: 95 | Primer for PRODH |
| SEQ ID NO: 96 | Primer for PRODH |
| SEQ ID NO: 97 | Primer for PRODH |
| SEQ ID NO: 98 | Primer for PRODH |
| SEQ ID NO: 99 | Primer for ANKK1 |
| SEQ ID NO: 100 | Primer for ANKK1 |
| SEQ ID NO: 101 | Primer for ANNK1 |
| SEQ ID NO: 102 | Primer for DISC1 |
| SEQ ID NO: 103 | Primer for DISC1 |
| SEQ ID NO: 104 | Primer for DISC1 |
| SEQ ID NO: 105 | Primer for GRM3 |
| SEQ ID NO: 106 | Primer for GRM3 |
| SEQ ID NO: 107 | Primer for GRM3 |
| SEQ ID NO: 108 | Primer for RGS4 |
| SEQ ID NO: 109 | Primer for RGS4 |
| SEQ ID NO: 110 | Primer for RGS4 |
| SEQ ID NO: 111 | Primer for CNR1 |
| SEQ ID NO: 112 | Primer for CNR1 |

TABLE 2
Individual SNP genotype and allele frequency association with schizophrenia

| | | | P value | P value | | | HWE |
|---|---|---|---|---|---|---|---|
| | SNP | | Allele | Genotype | Odds | | C = controls |
| SNP ID | details | GENE | Freq | Freq | Ratio | 95% CI | S = schizophrenics |
| Rs6277 | C957T | DRD2 | 0.0045 | 0.013 | 1.5085 | 1.1349-2.0052 | |
| Rs9370822 | Tag SNP | DTNBP1 | 0.0126 | 0.015 | 1.4537 | 1.0828-1.9517 | |
| | A/C | | | | | | |
| Rs1997679 | Tag SNP | DTNBP1 | 0.0315 | 0.048 | 1.3988 | 1.0295-1.9008 | |
| | C/T | | | | | | |
| Rs4263535 | Tag SNP | GABRA1 | 0.0060 | 0.017 | 1.6798 | 1.1573-2.4384 | |
| | A/G | | | | | | |
| Rs40184 | Tag SNP | DAT | 0.0064 | 0.022 | 1.4802 | 1.1158-1.9636 | |
| | A/G | | | | | | |
| Rs2975292 | Tag SNP | DAT | 0.0018 | 0.0047 | 1.5965 | 1.1885-2.1446 | |
| | C/G | | | | | | |
| Rs13161905 | Tag SNP | DAT | 0.0045 | 0.0196 | 1.5274 | 1.1389-2.0483 | |
| | C/T | | | | | | |
| Rs11133767 | Tag SNP | DAT | 0.0206 | 0.0056 | 1.4386 | 1.0565-1.959 | |
| | A/G | | | | | | |
| Rs4680 | MetI58Val | COMT | 0.0051 | 0.0146 | 1.4981 | 1.1283-1.9889 | |
| Rs165774 | Tag SNP | COMT | 0.0075 | 0.0076 | 1.5247 | 1.1178-2.0797 | |
| | A/G | | | | | | |
| Rs4975646 | Tag SNP | DAT | 0.0105 | 0.0313 | 1.1484 | 1.0959-2.0088 | |
| | A/G | | | | | | |
| Rs2842030 | Tag SNP | RGS4 | 0.0379 | 0.088 | 1.3567 | 1.0168-1.8103 | C = no; S = yes |
| | G/T | | | | | | |
| Rs9562919 | Tag SNP | KPNA3 | 0.0485 | 0.150 | 1.3365 | 1.0015-1.7836 | C = yes S = yes |
| | A/T | | | | | | |
| Rs3001371 | Tag SNP | AKT1 | 0.0301 | 0.015 | 1.4197 | 1.0335-1.9501 | C = no; S = yes |
| | C/T | | | | | | |
| Rs2770297 | Tag SNP | HTR2A | 0.0055 | 0.027 | 1.6633 | 1.1592-2.3866 | C = no; S = yes |
| | C/T | | | | | | |
| Rs5747933 | Asn275Thr | PRODH | 0.0181 | 0.046 | 2.2741 | 1.1311-4.572 | C = yes; S = no |
| Rs2238733 | Tag A/C | PRODH | 0.0416 | 0.042 | 1.4354 | 1.0128-2.0344 | C = no S = yes |
| Rs2870984 | Met466Thr | PRODH | 0.0009 | 0.007 | 2.2331 | 1.3765-3.6232 | C = no S = no |
| Rs1800497 | Taq1 A | ANKK1 | 0.0254 | 0.001 | 1.4895 | 1.049-2.1149 | C = no S = yes |
| Rs6675281 | Phe607Leu | DISC1 | 0.0557 | 0.0898 | 1.5297 | 1.0127-2.3702 | |
| Rs2214653 | Tag SNP | GRM3 | 0.0578 | 0.086 | 1.3431 | 0.9899-1.8223 | C = no; S = yes |
| | A/G | | | | | | |
| Rs10759 | Tag SNP | RGS4 | 0.0504 | 0.123 | 1.3643 | 1.001-1.8629 | C = yes; S = yes |
| | A/C | | | | | | |
| Rs1049353 | Tag SNP | CNR1 | 0.0460 | 0.131 | 1.38 | 0.99-1.93 | |
| | G/A | | | | | | |

TABLE 3
Schizophrenia Discrimination Panel

| | Improvement | | | Model | | | Correct Class % | |
|---|---|---|---|---|---|---|---|---|
| Step^{A} | Chi-square | df | Sig. | Chi-square | df | Sig. | Discrimination^{B} | SNP |
| 1 | 10.843 | 1 | .001 | 10.843 | 1 | .001 | 62.2% | RS2975292 |
| | | | | | | | | DAT |
| 2 | 8.824 | 1 | .003 | 19.667 | 2 | .000 | 62.9% | RS2870984 |
| | | | | | | | | PRODH |
| 3 | 9.757 | 1 | .002 | 29.424 | 3 | .000 | 66.6% | RS6277 |
| | | | | | | | | DRD2 |
| 4 | 8.005 | 1 | .005 | 37.429 | 4 | .000 | 68.5% | RS4680 |
| | | | | | | | | COMT |
| 5 | 7.873 | 1 | .005 | 45.302 | 5 | .000 | 68.5% | RS2770297 |
| | | | | | | | | HTR2A |
| 6 | 7.068 | 1 | .008 | 52.370 | 6 | .000 | 69.0% | RS4263535 |
| | | | | | | | | GABRA1 |
| 7 | 6.899 | 1 | .009 | 59.269 | 7 | .000 | 71.2% | RS40184 |
| | | | | | | | | DAT |
| 8 | 6.450 | 1 | .011 | 65.719 | 8 | .000 | 72.2% | RS3001371 |
| | | | | | | | | AKT1 |
| 9 | 5.693 | 1 | .017 | 71.412 | 9 | .000 | 71.2% | RS11913840 |
| | | | | | | | | PRODH |
| 10 | 5.110 | 1 | .024 | 76.522 | 10 | .000 | 71.0% | RS1800497 |
| | | | | | | | | ANKK1 |
| 11 | 4.967 | 1 | .026 | 81.488 | 11 | .000 | 72.0% | RS5747933 |
| | | | | | | | | PRODH |
| 12 | 4.766 | 1 | .029 | 86.254 | 12 | .000 | 72.4% | RS2238733 |
| | | | | | | | | PRODH |
| 13 | 5.160 | 1 | .023 | 91.414 | 13 | .000 | 72.2% | RS9370822 |
| | | | | | | | | DTNBP1 |

Step^{A} = Number of SNPs added stepwise in the panel
Discrimination^{B} = Discrimination (%) with the addition of each SNP

TABLE 4
Allele distribution of control and schizophrenia patients

| | | Distribution | | | |
|---|---|---|---|---|---|
| SNP | Gene | (%) | Alleles | P | OR |
| Rs2870984 | PRODH | G | A | 0.0009 | G |
| Patients | | 288 | 24 | | 2.2331 |
| Controls | | 403 | 75 | | |
| Rs2842030 | RGS4 | G | T | 0.0379 | G |
| Patient | | 149 | 163 | | 1.3567 |
| Control | | 190 | 282 | | |
| Rs2214653 | GRM3 | G | A | 0.0578 | A |
| Patient | | 167 | 123 | | 1.3431 |
| Control | | 279 | 153 | | |
| Rs10759 | RGS4 | C | A | 0.0504 | C |
| Patient | | 208 | 90 | | 1.3643 |
| Control | | 288 | 170 | | |
| Rs9562919 | KPNA3 | A | T | 0.049 | T |
| Patient | | 156 | 150 | | 1.3365 |
| Control | | 278 | 200 | | |
| Rs3001371 | AKT1 | C | T | 0.0301 | T |
| Patient | | 192 | 100 | | 1.4197 |
| Control | | 338 | 124 | | |
| Rs2770297 | HTR2A | T | C | 0.0055 | T |
| Patient | | 194 | 60 | | 1.6633 |
| Control | | 243 | 125 | | |
| Rs5747933 | PRODH | G | T | 0.0181 | T |
| Patient | | 294 | 20 | | 2.2741 |
| Control | | 468 | 14 | | |
| Rs2238733 | PRODH | C | A | 0.0416 | A |
| Patient | | 211 | 77 | | 1.4354 |
| Control | | 354 | 90 | | |
| Rs1800497 | ANK11 | C | T | 0.0254 | T |
| Patient | | 217 | 77 | | 1.4895 |
| Control | | 361 | 86 | | |
| Rs6277 | DRD2 | C | T | 0.0045 | C |
| Patient | | 170 | 144 | | 1.5085 |
| Control | | 216 | 276 | | |
| Rs9370822 | DTNBP1 | A | C | 0.0126 | C |
| Patient | | 189 | 129 | | 1.4537 |
| Control | | 328 | 154 | | |
| Rs1997679 | DTNBP1 | C | T | 0.0315 | C |
| Patient | | 231 | 89 | | 1.3988 |
| Control | | 321 | 173 | | |
| Rs4263535 | GABRA1 | G | A | 0.0060 | G |
| Patient | | 67 | 253 | | 1.6798 |
| Control | | 67 | 425 | | |
| Rs40184 | DAT | G | A | 0.0064 | A |
| Patient | | 154 | 166 | | 1.4802 |
| Control | | 287 | 209 | | |
| Rs2975292 | DAT | C | G | 0.0018 | G |
| Patient | | 190 | 130 | | 2.1446 |
| Control | | 343 | 147 | | |
| Rs13161905 | DAT | C | T | 0.0045 | C |
| Patient | | 211 | 107 | | 1.5274 |
| Control | | 275 | 213 | | |
| Rs11133767 | DAT | G | A | 0.0206 | A |
| Patient | | 212 | 108 | | 1.4386 |
| Control | | 353 | 125 | | |
| Rs4680 | COMT | G | A | 0.0051 | G |
| Patient | | 169 | 149 | | 1.4981 |
| Control | | 212 | 280 | | |
| Rs165774 | COMT | A | G | 0.0075 | G |
| Patient | | 84 | 232 | | 1.5247 |
| Control | | 175 | 317 | | |
| Rs4975646 | DAT | G | A | 0.0105 | G |
| Patient | | 228 | 92 | | 1.4837 |
| Control | | 309 | 185 | | |
| Rs6675281 | DISC1 | C | T | 0.0557 | C |
| Patient | | 287 | 33 | | 1.5298 |
| Control | | 415 | 73 | | |
| Rs1049353 | CNR1 | G | A | 0.046 | G |
| Patient | | 239 | 81 | | 1.38 |
| Control | | 454 | 145 | | |

The following gene identities are used in the specification (as described in Table 4).

TABLE 5
Gene Name

| Gene ID | Gene Name |
|---|---|
| AKT1 | Protein kinase B |
| ANKK1 | Ankyrin repeat and protein kinase domain-containing |
| | protein 1 |
| COMT | Catechol-O-methyl transferase |
| DAT | Dopamine associated transporter |
| DISC1 | Disrupted in schizophrenia |
| DRD2 | Dopamine D2 receptor |
| DTNBP1 | Dystrobrevin binding protein 1 (Dysbindin) |
| GABRA1 | Gamma-aminobutyric acid (GABA) A receptor alpha 1 |
| GRM3 | Glutamate receptor metabotropic 3 |
| HTR2A | 5-hydroxytryptamine (serotonin) receptor 2A |
| KPNA3 | Karyopherin alpha 3 (importin alpha 3) |
| PRODH | Proline dehydrogenase (oxidase) 1 |
| RGS4 | Regulator of G-protein signalling 4 |
| CNR1 | Cannabinoid Receptor 1 |

### DETAILED DESCRIPTION OF THE PREFERRED EMBODIMENT

The singular forms “a”, “an”, and “the” include single and plural aspects unless the context clearly indicates otherwise. Thus, for example, reference to “a polymorphism” includes a single polymorphism, as well as two or more polymorphisms; reference to “an association” includes a single association or multiple associations; reference to “the psychological phenotype” includes a single psychological phenotype, as well as two or more psychological phenotypes, and so on. In addition, reference to “the invention” includes single or multiple aspects of an invention.

The present invention is predicated in part on the identification of genetic profiles having a statistically significant association with a neurological, psychiatric or psychological condition, phenotype or state including a sub-threshold neurological, psychiatric or psychological condition, phenotype or state. However, the genetic profile is an indicator of an underlying biochemical or metabolic state that is responsible for the neurological, psychiatric or psychological condition, phenotype or state including a sub-threshold neurological, psychiatric or psychological condition, phenotype or state. By “genetic profiles” is meant that groups of individuals exhibiting a particular neurological, psychiatric or psychological condition, phenotype or state or sub-threshold forms thereof or who are at the risk of developing same exhibit a common polymorphism at or within one or genes selected from the list in Table 2 including its 5′ or 3′ terminal regions, promoter, exons or introns. The genetic profile may be a single polymorphism or multiple polymorphisms in a single gene or in a panel of genes, that is two or more polymorphisms in one or more genes that are statistically significantly linked to a neurological, psychiatric or psychological condition, phenotype or state or sub-threshold forms thereof. Reference to a polymorphism in this context includes a mutation. A mutation includes and is encompassed by the term “polymorphism”, a nucleotide insertion, addition, substitution and deletion as well as a rearrangement or microsatellite.

In a particular embodiment, the genetic profile comprises from about one to about 15 genes as listed in Table 2 such as 1, 2, 3, 4, 5, 6, 7, 8, 9, 10, 11, 12, 13, 14 or 15 genes. More particularly, the genetic profile comprises one or more polymorphisms in one or more of the 9 genes as defined in Table 3. The profile may be, therefore, a panel of polymorphisms. A given gene may also contain more than one polymorphism or there may be a polymorphism in each gene. Hence, the present invention extends to the identification of from about one to about 100 polymorphisms in one or more genes.

Although the genes shown in Table 3 are given a ranking this should in no way limit the diagnostic method of determining a mutation in these genes in any particular order. It is important to note that the present invention extends to a single gene in Table 2 or 3 or two or more genes in Table 2 or 3. With respect to the ranking in Table 3, again, all genes may be considered or a combination of two or more may be used.

Accordingly, one aspect of the present invention contemplates a method for identifying a genetic profile associated with a neurological, psychiatric or psychological condition, phenotype or state including a sub-threshold neurological, psychiatric or psychological condition, phenotype or state in an individual or a group of individuals, said method comprising screening individuals for a polymorphism including a mutation in a gene selected from the list in Table 2, including its 5′ and 3′ terminal regions, promoter, introns and exons which has a statistically significant linkage or association to symptoms or behavior characterizing the neurological, psychiatric or psychological condition, phenotype or state or sub-threshold forms thereof.

The genetic locus comprising the genes listed in Tables 2 and 3 may be referred to as the “gene”, “nucleic acid”, “locus”, “genetic locus” or “polynucleotide”. Each refers to polynucleotides, all of which are in the gene region including its 5′ or 3′ terminal regions, promoter, introns or exons. Accordingly, the genes of the present invention are intended to include coding sequences, intervening sequences and regulatory elements controlling transcription and/or translation. A genetic locus is intended to include all allelic variations of the DNA sequence on either or both chromosomes. Consequently, homozygous and heterozygous variations of the instant genetic loci are contemplated herein.

As indicated above, the present invention provides a genetic panel comprising different profiles of genes or mutations therein for different neurological, psychiatric or psychological conditions, phenotypes or states or sub-threshold forms thereof. Such profiles include polymorphisms, although any nucleotide substitution, addition, deletion or insertion or other mutation in one or more genetic loci is encompassed by the present invention when associated with a neurological, psychiatric or psychological condition, phenotype or state. Accordingly, the present invention extends to rare mutations which although not present in larger numbers of individuals in a population, when the mutation is present, it leads to a very high likelihood of development of a pathopsychological disorder. The present invention is not to be limited to all the genes in the genetic panel. A single gene or two or more genes in Table 2 or the entire panel or one and more particularly two or more genes in Table 3 may be used in accordance with the present invention.

The term “polymorphism” or “mutation” refers to a difference in a DNA or RNA sequence or sequences among individuals, groups or populations which give rise to a statistically significant phenotype or physiological condition. Examples of genetic polymorphisms include mutations that result by chance or are induced by external features. These polymorphisms or mutations may be indicative of a disease or disorder and may arise following a genetic disease, a chromosomal abnormality, a genetic predisposition, a viral infection, a fungal infection, a bacterial infection or a protist infection or following chemotherapy, radiation therapy or substance abuse including alcohol or drug abuse. The polymorphisms may also dictate or contribute to symptoms with a psychological phenotype. In a preferred aspect, the polymorphisms of the present invention are indicative of a neurological, psychiatric or psychological condition, phenotype or state or sub-threshold condition, phenotype or state thereof. As used herein, polymorphisms including mutations may refer to one or more changes in a DNA or RNA sequence which are present in a group of individuals having a particular neurological, psychiatric or psychological condition, phenotype or state or sub-threshold forms thereof or are at risk of developing same.

Examples of nucleotide changes contemplated herein include single nucleotide polymorphisms (SNPs), multiple nucleotide polymorphisms (MNPs), frame shift mutations, including insertions and deletions (also called deletion insertion polymorphisms or DIPS), nucleotide substitutions, nonsense mutations, rearrangements and microsatellites. Two or more polymorphisms may also be used either at the same allele (i.e. haplotypes) or at different alleles. All these mutations are encompassed by the term “polymorphism”.

Examples of a neurological, psychiatric or psychological condition, phenotype or state contemplated by the present invention and which may be directly or indirectly linked to a genetic profile such as a polymorphism or mutation related to dopamine pathway function and genes that influence the function of associated neurotransmitters GABA, glutamate, serotonin including but are not limited to addiction, dementia, anxiety disorders, bipolar disorder, schizophrenia, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia. It should be noted, however, that a person considered not to suffer any symptom associated with the above disorders still falls within the scope of a “normal” or a non-symptomatic or non-pathogenic neurological, psychiatric or psychological condition, phenotype or state.

Exemplified conditions herein are schizophrenia, anxiety disorders, nicotine dependence, alcohol dependence and opioid dependence. Reference herein to “schizophrenia” includes conditions which have symptoms similar to schizophrenia and hence are regard as schizophrenia-related conditions. Such symptoms of schizophrenia include behavioral and physiological conditions. A related condition may also have a common underlying genetic cause or association and/or a common treatment rationale. Due to the composition of schizophrenia and related conditions, the ability to identify a genetic profile to assist in defining schizophrenia is of significant importance. The present invention now provides this genetic profile. Further identification of potential genetic profiles may include a predisposition to developing a neurological, psychiatric or psychological condition, phenotype or state selected from addiction, dementia, anxiety disorders, bipolar disorder, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, and social phobia.

Any number of methods may be used to calculate the statistical significance of a polymorphism and its association with a neurological, psychiatric or psychological condition. Particular statistical analysis methods which may be used are described in Fisher and vanBelle, “Biostatistics: A Methodology for the Health Sciences” Wiley-Intersciences (New York) 1993. This analysis may also include a regression calculation of which polymorphic sites in the gene profile which gives the most significant contribution to the differences in phenotype. One regression model useful in the invention starts with a model of the form

r=r₀+(S×d)

where r is the response, r0 is a constant called the “intercept”, S is the slope and d is the dose. To determine the dose, the most-common and least common nucleotides at the polymorphic site are first defined. Then, for each individual in the trial population, one calculates a “dose” as the number of least-common nucleotides the individual has at the polymorphic site of interest. This value can be 0 (homozygous for the least-common nucleotide), 1 (heterozygous), or 2 (homozygous for the most common nucleotide). An individual's “response” is the value of the clinical measurement. Standard linear regression methods are then used to fit all the individuals' doses and responses to a single model (see e.g. Fisher and vanBelle, supra, Ch 9). The outputs of the regression calculation are the intercept r0, the slope S, and the variance (which measures how well the data fits this simple linear model). The Students t-test value and the level of significance can then be calculated for each of the polymorphic sites.

In relation to the genetic profile associated with schizophrenia, alcoholism or a related condition or other neurological, psychiatric or psychological condition, phenotype or state, the present invention encompasses a ranking comprising a polymorphism or mutation in a particular group of genes such as from about one to about 100 polymorphisms including the SNPs exemplified in Table 2. More particularly four to 15 genes may be used. In a particular embodiment, the ranking comprises the genes listed in Table 3 or a combination of two or more of the genes. Reference to “gene” includes its 5′ or 3′ terminal regions, promoter, introns and exons.

The present invention provides a genetic marker set for a neurological, psychiatric or psychological condition, state or phenotype in an individual wherein the genetic marker is selected from about one to about 100 polymorphisms in one or more genes as listed in Table 2 or more particularly in one or more genes listed in Table 3. It is proposed that these polymorphisms are indicative of, or a predisposition of developing a neurological, psychiatric or psychological condition, phenotype or state selected from addiction, dementia, anxiety disorders, bipolar disorder, schizophrenia, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia. It should be noted, however, that a person considered not to suffer any symptom associated with the above disorders still falls within the scope of a “normal” or a non-symptomatic or non-pathogenic neurological, psychiatric or psychological condition, phenotype or state.

In one embodiment, the neurological, psychiatric or psychological condition, phenotype or state is schizophrenia or alcoholism or a related condition.

In a particular embodiment the condition is schizophrenia or a related condition.

Accordingly, another aspect of the present invention provides a panel of genetic mutations providing a genetic marker set for schizophrenia or a related condition in an individual said genetic marker comprising from about one to about 100 polymorphisms in one or more genes listed in Table 2 wherein the presence of the polymorphisms is indicative of or a predisposition to developing.

In a related aspect, the present invention provides a method for detecting the presence of, or the propensity to develop a neurological, psychiatric or psychological condition phenotype or state or sub-threshold form thereof, wherein the condition, phenotype or state results from or is exacerbated by any insertion or deletion at the site of a polymorphism in a gene selected from Table 2 or more particularly in the genes selected in Table 3 including its 5′ or 3′ terminal regions, promoter, exons or introns. Insertions or deletions may involve a single nucleotide or more than one such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides within the region of interest. Rearrangements, microsatellites and other nucleotide insertions, additions, substitutions or deletions may also occur.

In yet another aspect, the present invention provides a nonsense mutation which includes the introduction of a stop codon.

A neurological, psychiatric or psychological condition, phenotype or state or sub-threshold form thereof involving one or more genes from Table 2 or Table 3 or a risk of developing such a condition, phenotype or state may be ascertained by screening any tissue from an individual for genetic material carrying the genetic locus for the presence of a polymorphism including a mutation which is associated with a particular neurological, psychiatric or psychological condition, phenotype or state or a sub-threshold form thereof or a pre-disposition for development of same. Schizophrenia is an example of a particular neurological, psychiatric or psychological condition, phenotype or state. Most conveniently, buccal cells are obtained or blood is drawn and DNA extracted from the cells. In addition, prenatal diagnosis can be accomplished by testing foetal cells, placental cells or amniotic cells for a polymorphism in one or more genes to detect the presence of a genetic profile comprising from about one to about 100 polymorphisms such as the SNPs exemplified in Table 2 or more particularly in one or more genes from Table 3.

Accordingly, another aspect of the present invention contemplates a method for diagnosing a neurological, psychiatric or psychological condition, phenotype or state in an individual, said method comprising obtaining or extracting DNA sample from cells of said individual and screening for or otherwise detecting the presence of a genetic profile comprising from about one to about 100 polymorphisms in one or more genes listed in Table 2 such as a ranking of two or more polymorphisms in the genes listed in Table 3 with a statistically significant association with a particular neurological, psychiatric or psychological condition, phenotype or state wherein the presence of that genetic profile is indicative of the neurological, psychiatric or psychological condition, phenotype or state or a sub-threshold form thereof or that the individual is at risk of developing same.

Generally, the genetic test is part of an overall diagnostic protocol involving clinical assessment and diagnostic tools such as pencil-and-paper tests. Consequently, this aspect of the present invention may be considered as a confirmatory test or part of a series of tests in the final diagnosis of a neurological, psychiatric or psychological condition, phenotype or state.

Accordingly, another aspect of the present invention provides a diagnostic assay for a genetic profile predetermined to be associated with a particular neurological, psychiatric or psychological condition, phenotype or state said method comprising obtaining or extracting a DNA sample from cells of said individual and screening for or otherwise detecting the presence of from about one to about 100 polymorphisms in one or more genes listed in Table 2 such as a ranking of two or more polymorphisms in one or more genes listed in Table 3 which has a statistically significant association with a particular neurological, psychiatric or psychological condition, phenotype or state wherein the presence of that genetic profile is indicative of the neurological, psychiatric or psychological condition, phenotype or state or a sub-threshold form thereof or that the individual is at risk of developing same.

As indicated above, the genetic profile is generally detecting polymorphisms in a range of genes ranked in order of statistically significance in its association with its disorder. Any polymorphism or mutation such as those contemplated in Tables 1 and 2 and which are found to be associated with a neurological, psychiatric or psychological condition, phenotype or state is encompassed by the present invention. In addition, examples of neurological, psychiatric or psychological conditions, phenotypes and states include but are not limited to addiction, dementia, anxiety disorders, bipolar disorder, schizophrenia, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia. It should be noted, however, that a person considered not to suffer any symptom associated with the above disorders still falls within the scope of a “normal” or a non-symptomatic or non-pathogenic neurological, psychiatric or psychological condition, phenotype or state.

Schizophrenia, anxiety disorder, and alcohol dependence, nicotine dependence and opioid dependence are particularly contemplated by the present invention.

Accordingly, in a preferred embodiment, the present invention is directed to a method for diagnosing a neurological, psychiatric or psychological condition, phenotype or, state including schizophrenia in an individual or a risk of development of same, said method comprising obtaining or extracting a DNA sample from cells of said individual and screening for or otherwise detecting the presence of a polymorphism in cDNA molecule corresponding to from one to 15 genes in Table 2 such two or more genes in Table 3 wherein the presence of a set of polymorphisms in the one to 15 genes is indicative of the individual having or at risk of developing an adverse neurological, psychiatric or psychological condition, phenotype or state selected from addiction, dementia, anxiety disorders, bipolar disorder, schizophrenia, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia.

The method and assay of the present invention are further directed to detecting the form of the polymorphism in an individual associated with “normal” behavior. In other words, an individual which may be at risk such as through his or her genetic lines or because of substance abuse or who has behavioral tendencies which suggest a particular neurological, psychiatric or psychological condition, phenotype or state can be screened for the presence of a polymorphism such as from about one to about 100 polymorphisms in from one to 15 genes as listed in Table 2 such as two or more polymorphisms in one or more genes in Table 3 wherein the presence of the profile of polymorphisms is at least suggestive of a genetic basis for any symptoms associated with the neurological, psychiatric or psychological condition, phenotype or state for which the individual first presented to a clinician.

Reference to “1 to 15 genes” includes 1, 2, 3, 4, 5, 6, 7, 8, 9, 10, 11, 12, 13, 14 or 15 genes.

A “neurological, psychiatric or psychological condition, phenotype or state” may be an adverse condition or may represent “normal” behavior. The latter constitutes behavior consistent with societal “norms”.

Reference herein to an “individual” includes a human which may also be considered a subject, patient, host, recipient or target.

The present invention enables, therefore, a stratification of individuals based on a genetic profile. The stratification or profiling enables early diagnosis, conformation of a clinical diagnosis, treatment monitoring and treatment selection for a neurological, psychiatric or psychological conditions phenotype or state.

There are many methods which may be used to detect a DNA sequence profile. Direct DNA sequencing, either manual sequencing or automated fluorescent sequencing can detect sequence variation including a polymorphism or mutation. Another approach is the single-stranded conformation polymorphism assay (SSCP) (Orita, et al, Proc. Natl. Acad. Sci. USA. 86:2766-2770, 1989). This method does not detect all sequence changes, especially if the DNA fragment size is greater than 200 bp, but can be optimized to detect most DNA sequence variation. The reduced detection sensitivity is a disadvantage, but the increased throughput possible with SSCP makes it an attractive, viable alternative to direct sequencing for mutation detection. The fragments which have shifted mobility on SSCP gels are then sequenced to determine the exact nature of the DNA sequence variation. Other approaches based on the detection of mismatches between the two complementary DNA strands include clamped denaturing gel electrophoresis (CDGE) (Sheffield et al, Proc. Natl. Acad. Sci. USA 86:232-236, 1989), heteroduplex analysis (HA) (White et al, Genomics 12:301-306, 1992) and chemical mismatch cleavage (CMC) (Grompe et al, Proc. Natl. Acad. Sci. USA 86:5855-5892, 1989). None of the methods described above detects large deletions, duplications or insertions, nor will they detect a mutation in a regulatory region or a gene. Other methods which would detect these classes of mutations include a protein truncation assay or the asymmetric assay. A review of currently available methods of detecting DNA sequence variation can be found in Kwok (Curr Issues Mol. Biol. 5(2):43-60, 2003, Twyman and Primrose (Pharmacogenomics. 4(1):67-79, 2003), Edwards and Bartlett (Methods Mol. Biol. 226:287-294, 2003) and Brennan (Am. J. Pharmacogenomics. 1(4):395-302, 2001). Once a mutation is known, an allele-specific detection approach such as allele-specific oligonucleotide (ASO) hybridization can be utilized to rapidly screen large numbers of other samples for that same mutation. Such a technique can utilize probes which are labeled with gold nanoparticles or any other reporter molecule to yield a visual color result (Elghanian et al, Science 277:1078-1081, 1997).

A rapid preliminary analysis to detect polymorphisms in DNA sequences can be performed by looking at a series of Southern blots of DNA cut with one or more restriction enzymes, preferably with a large number of restriction enzymes. Each blot contains a series of normal individuals and a series of individuals having neurologic or neuropsychiatric diseases or disorders or any other neurological, psychiatric or psychological condition, phenotype or state. Southern blots displaying hybridizing fragments (differing in length from control DNA when probed with sequences near or to the genetic locus being tested) indicate a possible mutation or polymorphism. If restriction enzymes which produce very large restriction fragments are used, then pulsed field gel electrophoresis (PFGE) is employed. Alternatively, the desired region of the genetic locus being tested can be amplified, the resulting amplified products can be cut with a restriction enzyme and the size of fragments produced for the different polymorphisms can be determined.

Detection of point mutations may be accomplished by molecular cloning of the target genes and sequencing the alleles using techniques well known in the art. Also, the gene or portions of the gene may be amplified, e.g., by PCR or other amplification technique, and the amplified gene or amplified portions of the gene may be sequenced.

Methods for a more complete, yet still indirect, test for confirming the presence of a susceptibility allele include: 1) single-stranded conformation analysis (SSCP) (Orita et al, supra 1989); 2) denaturing gradient gel electrophoresis (DGGE) (Wartell et al, Nucl. Acids Res. 18:2699-2705, 1990; Sheffield et al, supra 1989); 3) RNase protection assays (Finkelstein et al, Genomics 7:167-172, 1990; Kinszler et al, Science 251:1366-1370, 1991); 4) allele-specific oligonucleotides [ASOs] (Conner et al, Proc. Natl. Acad. Sci. USA 80:278-282, 1983); 5) the use of proteins which recognize nucleotide mismatches, such as the E. coli mutS protein (Modrich Ann. Rev. Genet. 25:229-253, 1991); 6) allele-specific PCR (Ruano and Kidd, Nucl. Acids Res. 17:8392, 1989); and 7) PCR amplification of the site of the polymorphism followed by digestion using a restriction endonuclease that cuts or fails to cut when the variant allele is present.

Additionally, real-time PCR such as the allele specific kinetic real-time PCR assay can be used or allele specific real-time TaqMan probes.

For allele-specific PCR, primers are used which hybridize at their 3′ ends to a particular target genetic locus or mutation. If the particular polymorphism or mutation is not present, an amplification product is not observed. Amplification Refractory Mutation System (ARMS) can also be used, as disclosed in European Patent Application Publication No. 0332435. Insertions and deletions of genes can also be detected by cloning, sequencing and amplification. In addition, restriction fragment length polymorphism (RFLP) probes for the gene or surrounding marker genes can be used to score alteration of an allele or an insertion in a polymorphic fragment. Such a method is particularly useful for screening relatives of an affected individual for the presence of the mutation found in that individual. Other techniques for detecting insertions and deletions as known in the art can be used.

In SSCP, DGGE and the RNase protection assay, an electrophoretic band appears which is absent if the polymorphism or mutation is not present. SSCP detects a band which migrates differentially because the sequence change causes a difference in single-strand, intramolecular base pairing. RNase protection involves cleavage of the mutant polynucleotide into two or more smaller fragments. DGGE detects differences in migration rates of mutant sequences compared to wild-type sequences, using a denaturing gradient gel. In an allele-specific oligonucleotide assay, an oligonucleotide is designed which detects a specific sequence, and the assay is performed by detecting the presence or absence of a hybridization signal, In the mutS assay, the protein binds only to sequences that contain a nucleotide mismatch in a heteroduplex between mutant and wild-type sequences.

Mismatches, according to the present invention, are hybridized nucleic acid duplexes in which the two strands are not 100% complementary. Lack of total homology may be due to deletions, insertions, inversions or substitutions. Mismatch detection can be used to detect point mutations in the gene or in its mRNA product. While these techniques are less sensitive than sequencing, they are simpler to perform on a large number of samples. An example of a mismatch cleavage technique is the RNase protection method. In the practice of the present invention, the method involves the use of a labeled riboprobe which is complementary to the human wild-type genes (i.e. such as those listed in Table 2). The riboprobe and either mRNA or DNA isolated from the person are annealed (hybridized) together and subsequently digested with the enzyme RNase A which is able to detect some mismatches in a duplex RNA structure. If a mismatch is detected by RNase A, it cleaves at the site of the mismatch. Thus, when the annealed RNA preparation is separated on an electrophoretic gel matrix, if a mismatch has been detected and cleaved by RNase A, an RNA product will be seen which is smaller than the full length duplex RNA for the riboprobe and the mRNA or DNA. The riboprobe need not be the full length of the mRNA or gene but can be a segment of either. If the riboprobe comprises only a segment of the mRNA or gene, it will be desirable to use a number of these probes to screen the whole mRNA sequence for mismatches.

In similar fashion, DNA probes can be used to detect mismatches, through enzymatic or chemical cleavage (see, for example, Cotton et al, Proc. Natl. Acad. Sci. USA 87:4033-40371988; Shenk et al, Proc. Natl. Acad. Sci. USA 72:989-993, 1975; Novack et al, Proc. Natl. Acad. Sci. USA 83:586-590, 1986). Alternatively, mismatches can be detected by shifts in the electrophoretic mobility of mismatched duplexes relative to matched duplexes (see, for example, Cariello Am. J. Human Genetics 42:726-734, 1988). With either riboprobes or DNA probes, the cellular mRNA or DNA which might contain a mutation can be amplified using PCR (see below) before hybridization. Changes in DNA of the associated genetic polymorphisms or genetic loci can also be detected using Southern blot hybridization, especially if the changes are gross rearrangements, such as deletions and insertions.

DNA sequences of the DRD2, DTNBP1, GABRA1, DAT, COMT, RGS4, KPNA3, AKT1, HTR2A, PRODH, ANKK1, DISC1 and GRM3 genes which have been amplified by use of PCR may also be screened using allele-specific probes. These probes are nucleic acid oligomers, each of which contains a region of the gene sequence harboring a known mutation. For example, one oligomer may be from about three to about 100 nucleotides in length such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100. An oligomer of about 20 nucleotides in length is particularly convenient. These oligomers correspond to a portion of the gene sequence. By use of a battery of such allele-specific probes, PCR amplification products can be screened to identify the presence of a previously identified mutation in the gene. Hybridization of allele-specific probes with amplified target gene sequences can be performed, for example, on a nylon filter. Hybridization to a particular probe under high stringency hybridization conditions indicates the presence of the same mutation in the tissue as in the allele-specific probe.

Once the site containing the polymorphisms has been amplified, the SNPs can also be detected by primer extension. Here a primer is annealed immediately adjacent to the variant site, and the 5′ end is extended a single base pair by incubation with di-deoxytrinucleotides. Whether the extended base was a A, T, G or C can then be determined by mass spectrometry (MALDI-TOF) or fluorescent flow cytometric analysis (Taylor et al, Biotechniques 30:661-669, 2001) or other techniques.

Nucleic acid analysis via microchip technology is also applicable to the present invention. In this technique, thousands of distinct oligonucleotide probes are built up in an array on a silicon chip. Nucleic acids to be analyzed are fluorescently labeled and hybridized to the probes on the chip. It is also possible to study nucleic acid-protein interactions using these nucleic acid microchips. Using this technique, one can determine the presence of mutations or even sequence the nucleic acid being analyzed or one can measure expression levels of a gene of interest. The method is one of parallel processing of many, including thousands, of probes at once and can tremendously increase the rate of analysis.

The most definitive test for mutations in the target loci is to directly compare genomic sequences from patients with those from a control population. Alternatively, one can sequence mRNA after amplification, e.g., by PCR, thereby eliminating the necessity of determining the exon structure of the candidate gene.

Mutations falling outside the coding region of the target loci can be detected by examining the non-coding regions, such as introns and regulatory sequences near or within the genes. An early indication that mutations in non-coding regions are important may come from Northern blot experiments that reveal messenger RNA molecules of abnormal size or abundance in patients as compared to those of control individuals.

Alteration of mRNA expression from the genetic loci can be detected by any techniques known in the art. These include Northern blot analysis, PCR amplification and RNase protection. Diminished mRNA expression indicates an alteration of the wild-type gene. It is worth noting that the DRD2 957C>T polymorphism has been shown to increase mRNA stability in vitro (Duan et al, Hum Mol Genet. 12:205-16, 2003) and that this could result in a detectable change in steady-state DRD2 mRNA levels in vivo. Alteration of wild-type genes can also be detected by screening for alteration of wild-type protein. For example, monoclonal antibodies immunoreactive with a target protein (i.e. a protein encoded by a gene in Table 2 or two or more proteins from the genes in Table 2 or 3) can be used to screen a tissue. Lack of cognate antigen or a reduction in the levels of antigen would indicate a mutation. Antibodies specific for products of mutant alleles could also be used to detect mutant gene product. Such immunological assays can be done in any convenient formats known in the art. These include Western blots, immunohistochemical assays and ELISA assays. Any means for detecting an altered protein can be used to detect alteration of the wild-type protein. Functional assays, such as protein binding determinations, can be used. In addition, assays can be used which detect the protein biochemical function. Finding a mutant gene product indicates alteration of a wild-type gene product.

Hence, the present invention further extends to a method for identifying a genetic basis behind diagnosing or treating a neurological, psychiatric or psychological condition, phenotype or state in an individual, said method comprising obtaining a biological sample from said individual and detecting a protein encoded by a nucleotide sequence having from about one to about 100 polymorphisms in one or more genes listed in Table 2 including their 5′ or 3′ terminal region, promoter, intron or exons with a statistical significant association with a particular neurological, psychiatric or psychological condition, phenotype or state resulting in from about one to about 100 amino acid insertions, substitutions or deletions wherein the presence of an altered amino acid sequence is indicative of the presence of a polymorphism and the likelihood of a neurological, psychiatric or psychological condition, phenotype or psychological condition, phenotype or state.

The altered amino acid sequence may be detected via specific antibodies which can discriminate between the presence or absence of an amino acid change, by amino acid sequencing, by a change in protein activity or cell phenotype and/or via the presence of particular metabolites if the protein is associated with a biochemical pathway.

A mutant gene or corresponding gene products can also be detected in other human body samples which contain DNA, such as serum, stool, urine and sputum. The same techniques discussed above for detection of mutant genes or gene products in tissues can be applied to other body samples. By screening such body samples, an early diagnosis can be achieved for subjects at risk of developing a particular neurological, psychiatric or psychological condition, phenotype or state or sub-threshold forms thereof.

Primer pairs disclosed herein are useful for determination of the nucleotide sequence of a particular target gene using PCR. The pairs of single-stranded DNA primers can be annealed to sequences within or surrounding the gene in order to prime amplifying DNA synthesis of the gene itself. A complete set of these primers allows synthesis of all of the nucleotides of the gene coding sequences, i.e., the exons. The set of primers preferably allows synthesis of both intron and exon sequences. Allele-specific primers can also be used. Such primers anneal only to particular polymorphic or mutant alleles, and thus will only amplify a product in the presence of the polymorphic or mutant allele as a template.

In order to facilitate subsequent cloning of amplified sequences, primers may have restriction enzyme site sequences appended to their 5′ ends. Thus, all nucleotides of the primers are derived from the gene sequence or sequences adjacent the gene, except for the few nucleotides necessary to form a restriction enzyme site. Such enzymes and sites are well known in the art. The primers themselves can be synthesized using techniques which are well known in the art. Generally, the primers can be made using oligonucleotide synthesizing machines which are commercially available. Given the sequence of each gene and polymorphisms described herein, design of particular primers is well within the skill of the art. The present invention adds to this by presenting data on the intron/exon boundaries thereby allowing one to design primers to amplify and sequence all of the exonic regions completely.

The nucleic acid probes provided by the present invention are useful for a number of purposes. They can be used in Southern blot hybridization to genomic DNA and in the RNase protection method for detecting point mutations already discussed above. The probes can be used to detect PCR amplification products. They may also be used to detect mismatches in the target genes or mRNA using other techniques.

The present invention identifies the presence of altered (or mutant) genetic loci associated with a neurological, psychiatric or psychological condition, phenotype or state, including schizophrenia or a sub-threshold form thereof or an individual of risk of developing same. In order to detect a target genes or mutation, a biological sample is prepared and analyzed for a difference between the sequence of the allele being analyzed and the sequence of the “wild-type” allele. In this context, a “wild-type” allele includes the nucleotide at a given position most commonly represented in the population and for which there is not direct evidence for these individuals having the neurological, psychiatric or psychological condition, phenotype or state under investigation. Polymorphic or mutant alleles can be initially identified by any of the techniques described above. The polymorphic or mutant alleles may then be sequenced to identify the specific polymorphism or mutation of the particular allele. Alternatively, polymorphic or mutant alleles can be initially identified by identifying polymorphic or mutant (altered) proteins, using conventional techniques. The polymorphisms or mutations, especially those statistically associated with a neurological, psychiatric or psychological condition, phenotype or state or a sub-threshold form thereof are then used for the diagnostic and prognostic methods of the present invention.

As used herein, the phrase “amplifying” refers to increasing the content of a specific genetic region of interest within a sample. The amplification of the genetic region of interest may be performed using any method of amplification known to those of skill in the relevant art. In a preferred aspect, the present method for detecting a polymorphism utilizes PCR as the amplification step.

PCR amplification utilizes primers to amplify a genetic region of interest. Reference herein to a “primer” is not to be taken as any limitation to structure, size or function. Reference to primers herein, includes reference to a sequence of deoxyribonucleotides comprising at least three nucleotides. Generally, the primers comprises from about three to about 100 nucleotides, preferably from about five to about 50 nucleotides and even more preferably from about 10 to about 25 nucleotides such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. The primers of the present invention may be synthetically produced by, for example, the stepwise addition of nucleotides or may be fragments, parts or portions or extension products of other nucleic acid molecules. The term “primer” is used in its most general sense to include any length of nucleotides which, when used for amplification purposes, can provide free 3′ hydroxyl group for the initiation of DNA synthesis by a DNA polymerase. DNA synthesis results in the extension of the primer to produce a primer extension product complementary to the nucleic acid strand to which the primer has annealed or hybridized.

Accordingly, the present invention extends to an isolated oligonucleotide which comprises from about three to about 100 consecutive nucleotides from the gene or its corresponding cDNA or mRNA as listed in Table 2 such as the groups of two or more genes in Table 3 which encompass at least one polymorphism or mutation associated with or otherwise likely to be found in individuals with a particular neurological, psychiatric or psychological condition, phenotype or state such as those selected from normal behavior, addiction, dementia, anxiety disorders, bipolar disorder, schizophrenia, Tourette's syndrome, obsessive compulsive disorder (OCD), panic disorder, PTSD, phobias, acute stress disorder, adjustment disorder, agoraphobia without history of panic disorder, alcohol dependence (alcoholism), amphetamine dependence, brief psychotic disorder, cannabis dependence, cocaine dependence, cyclothymic disorder, delirium, delusional disorder, dysthymic disorder, generalized anxiety disorder, hallucinogen dependence, major depressive disorder, nicotine dependence, opioid dependence, paranoid personality disorder, Parkinson's disease, schizoaffective disorder, schizoid personality disorder, schizophreniform disorder, schizotypal personality disorder, sedative dependence, shared psychotic disorder, smoking dependence and social phobia. It should be noted, however, that a person considered not to suffer any symptom associated with the above disorders still falls within the scope of a “normal” or a non-symptomatic or non-pathogenic neurological, psychiatric or psychological condition, phenotype or state.

In a preferred embodiment, one of the at least two primers is involved in an amplification reaction to amplify a target sequence. If this primer is also labeled with a reporter molecule, the amplification reaction will result in the incorporation of any of the label into the amplified product. The terms “amplification product” and “amplicon” may be used interchangeably.

The primers and the amplicons of the present invention may also be modified in a manner which provides either a detectable signal or aids in the purification of the amplified product.

A range of labels providing a detectable signal may be employed. The label may be associated with a primer or amplicon or it may be attached to an intermediate which subsequently binds to the primer or amplicon. The label may be selected from a group including a chromogen, a catalyst, an enzyme, a fluorophore, a luminescent molecule, a chemiluminescent molecule, a lanthanide ion such as Europium (Eu³⁴), a radioisotope and a direct visual label. In the case of a direct visual label, use may be made of a colloidal metallic or non-metallic particular, a dye particle, an enzyme or a substrate, an organic polymer, a latex particle, a liposome, or other vesicle containing a signal producing substance and the like. A large number of enzymes suitable for use as labels is disclosed in U.S. Pat. Nos. 4,366,241, 4,843,000 and 4,849,338. Suitable enzyme labels useful in the present invention include alkaline phosphatase, horseradish peroxidase, luciferase, β-galactosidase, glucose oxidase, lysozyme, malate dehydrogenase and the like. The enzyme label may be used alone or in combination with a second enzyme which is in solution. Alternatively, a fluorophore which may be used as a suitable label in accordance with the present invention includes, but is not limited to, fluorescein-isothiocyanate (FITC), and the fluorochrome is selected from FITC, cyanine-2, Cyanine-3, Cyanine-3.5, Cyanine-5, Cyanine-7, fluorescein, Texas red, rhodamine, lissamine and phycoerythrin.

Examples of fluorophores are provided in Table 6.

TABLE 6

| | | |
|---|---|---|
| Probe | Ex¹ (nm) | Em² (nm) |

Reactive and conjugated probes

| | | |
|---|---|---|
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissamine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3, 5 | 581 | 596; (640) |
| Cy5 | (625); 650 | 670 |
| Cy5, 5 | 675 | 694 |
| Cy7 | 743 | 767 |

Nucleic acid probes

| | | |
|---|---|---|
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Mithramycin | 445 | 575 |
| YOYO-1 | 491 | 509 |
| SYTOX Green | 504 | 523 |
| SYTOX Orange | 547 | 570 |
| Ethidium Bromide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |

Cell function probes

| | | |
|---|---|---|
| Indo-1 | 361/330 | 490/405 |
| Fluo-3 | 506 | 526 |
| DCFH | 505 | 535 |
| DHR | 505 | 534 |
| SNARF | 548/579 | 587/635 |

Fluorescent Proteins

| | | |
|---|---|---|
| Y66F | 360 | 508 |
| Y66H | 360 | 442 |
| EBFP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |

Other probes

| | | |
|---|---|---|
| Monochlorobimane | 380 | 461 |
| Calcein | 496 | 517 |

¹Ex: Peak excitation wavelength (nm)
²Em: Peak emission wavelength (nm)

In order to aid in the purification of an amplicon, the primers or amplicons may additionally be incorporated on a bead. The beads used in the methods of the present invention may either be magnetic beads or beads coated with streptavidin.

The extension of the hybridized primer to produce an extension product is included herein by the term amplification. Amplification generally occurs in cycles of denaturation followed by primer hybridization and extension. The present invention encompasses form about one cycle to about 120 cycles, preferably from about two to about 70 cycles, more preferably from about five to about 40 cycles, including 10, 15, 20, 25 and 30 cycles, and even more preferably, 35 cycles such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 cycles.

In order for the primers used in the methods of the present invention to anneal to a nucleic acid molecule containing the gene of interest, a suitable annealing temperature must be determined. Determination of an annealing temperature is based primarily on the genetic make-up of the primer, i.e. the number of A, T, C and Gs, and the length of the primer. Annealing temperatures contemplated by the methods of the present invention are from about 40° C. to about 80° C., preferably from about 50° C. to about 70° C., and more preferably about 65° C. such as 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80° C.

The PCR amplifications performed in the methods of the present invention include the use of MgCl₂ in the optimization of the PCR amplification conditions. The present invention encompasses MgCl₂ concentrations for about 0.1 to about 10 mM, preferably from 0.5 to about 5 mM, and even more preferably 2.5 mM such as 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 or 10 mM.

Polymorphisms of the present invention may be detected due to the presence of a base mis-match in the heteroduplexes formed following PCR amplification. A base mis-match occurs when two nucleotide sequences are aligned with substantial complementarity but at least one base aligns to a base which would result in an “abnormal” binding pair. An abnormal binding pair occurs when thymine (T) were to bind to a base other than adenine (A), if A were to bind to a base other than T, if guanine (G) were to bind to a base other than cytosine (C) or if C was to bind to a base other than G.

In order to detect the presence of alleles from the genes from Table 2 or combination of two or more genes in Table 3 predisposing an individual to an inability to overcome a neurological, psychiatric or psychological condition, phenotype or state or sub-threshold form thereof or a risk of developing same, a biological sample such as blood is obtained and analyzed for the presence or absence of a panel of susceptibility alleles comprising from about one to 15 alleles of the genetic loci identified as being statistically significantly associated with the neurological, psychiatric or psychological condition, phenotype or state of interest. Results of these tests and interpretive information are returned to the health care provider for communication to the tested individual. Such diagnoses may be performed by diagnostic laboratories, or, alternatively, diagnostic kits are manufactured and sold to health care providers or to private individuals for self-diagnosis. Suitable diagnostic techniques include those described herein as well as those described in U.S. Pat. Nos. 5,837,492; 5,800,998 and 5,891,628.

According to the present invention, a method is also provided for supplying wild-type genes as listed in Tables 2 or 3 to a cell which carries a mutant or polymorphism. Supplying such a function should allow normal functioning of the recipient cells. The wild-type gene or a part of the gene may be introduced into the cell in a vector such that the gene remains extrachromosomal, in such a situation, the gene will be expressed by the cell from the extrachromosomal location. More preferred is the situation where the wild-type gene or a part thereof is introduced into the mutant cell in such a way that it recombines with the endogenous mutant gene present in the cell. Such recombination requires a double recombination event which results in the correction of the gene mutation. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner. Conventional methods are employed, including those described in U.S. Pat. Nos. 5,837,492; 5,800,998 and 5,891,628.

The identification of the association between a gene polymorphism/mutation and a psychological phenotype or sub-threshold psychological phenotype permits the early presymptomatic screening of individuals to identify those at risk for developing a neurological, psychiatric or psychological condition, phenotype or state or sub-threshold neurological, psychiatric or psychological condition, phenotype or state such as schizophrenia or to identify the cause of such disorders or the risk that any individual will develop same. To identify such individuals, the alleles are screened as described herein or using conventional techniques, including but not limited to, one of the following methods: fluorescent in situ hybridization (FISH), direct DNA sequencing, PFGE analysis, Southern blot analysis, single stranded conformation analysis (SSCP), linkage analysis, RNase protection assay, allele-specific oligonucleotide (ASO), dot blot analysis and PCR-SSCP analysis. Also useful is the recently developed technique of DNA microchip technology. Such techniques are described in U.S. Pat. Nos. 5,837,492; 5,800,998 and 5,891,628, each incorporated herein by reference.

Genetic testing enables practitioners to identify or stratify individuals at risk for certain behavioral states including substance addition or an inability to overcome a neurological, psychiatric or psychological condition, phenotype or state or a sub-threshold form thereof after initial treatment. For particular at risk couples, embryos or fetuses may be tested after conception to determine the genetic likelihood of the offspring being pre-disposed to the neurological, psychiatric or psychological condition, phenotype or state. Certain behavioral or therapeutic protocols may then be introduced from birth or early childhood to reduce the risk of the neurological, psychiatric or psychological condition, phenotype or state developing. Presymptomatic diagnosis will enable better treatment of these disorders, including the use of existing medical therapies. Genetic testing will also enable practitioners to identify individuals having diagnosed disorders (or in an at risk group) which have polymorphism identified in the genetic loci. Genotyping of such individuals will be useful for (a) identifying neurological, psychiatric or psychological condition, phenotype or state or a sub-threshold form thereof that will respond to drugs affecting gene product activity, (b) identifying a neurological, psychiatric or psychological condition, phenotype or state or sub-threshold neurological, psychiatric or psychological condition, phenotype or state that in an individual which respond well to specific medications or medication types with fewer adverse effects and (c) guide new drug discovery and testing.

Further, the present invention provides a method for screening drug candidates to identify molecules useful for treating neurological, psychiatric or psychological conditions, phenotypes or states involving the gene or its expression product. Drug screening is performed by comparing the activity of native genes and those described herein in the presence and absence of potential drugs. In particular, these drugs may have the affect of masking a polymorphism or mutation or may bind to a particular polymorphism or mutation enabling it to be used as a diagnostic agent. The terms “drug”, “agent”, “therapeutic molecule”, “prophylactic molecule”, “medicament”, “candidate molecule” or “active ingredient” may be used interchangeable in describing this aspect of the present invention.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo or which are specific for a targetable (e.g. a polymorphism) and hence is a useful diagnostic. Several approaches for use in rational drug design include analysis of three-dimensional structure, alanine scans, molecular modeling and use of anti-id antibodies. These techniques are well known to those skilled in the art, including those described in U.S. Pat. Nos. 5,837,492; 5,800,998 and 5,891,628.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal, Mimetic design, synthesis and testing are generally used to avoid randomly screening large numbers of molecules for a target property.

Once the pharmacophore has been found, its structure is modelled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process. A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Briefly, a method of screening for a substance which modulates activity of a polypeptide may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

Following identification of a substance which modulates or affects gene or gene product activity, the substance may be further investigated. Furthermore, it may be manufactured and/or used in preparation, i.e., a manufacture or formulation, or a composition such as a medicament, pharmaceutical composition or drug. These may be administered to individuals directly or via gene therapy.

The expression products of the genes in Table 2 or 3, antibodies, peptides and nucleic acids of the present invention can be formulated in pharmaceutical compositions, which are prepared according to conventional pharmaceutical compounding techniques. See, for example, Remington's Pharmaceutical Sciences, 18th Ed. 1990, Mack Publishing Co., Easton, Pa. The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g., intravenous, oral, intrathecal, epineural or parenteral.

The present invention provides information necessary for medical practitioners to select drugs for use in the treatment of a neurological, psychiatric or psychological condition, phenotype or state or a sub-threshold form thereof. With the identification that polymorphisms within a panel of genes are associated with a neurological, psychiatric or psychological condition, phenotype or state including a sub-threshold form thereof, such as schizophrenia, antipsychotic medications, can be selected for the treatment of such conditions.

The present invention further contemplates a method of treating a neurological, psychiatric or psychological condition, phenotype or state in an individual the method comprising identifying from about one to about 100 polymorphisms in a gene selected from Table 2 or a panel of genes selected from two or more genes in Table 3 with the neurological, psychiatric or psychological condition, phenotype or state and subjecting the individual to gene therapy to alter the gene or genetic sequence having a different polymorphism or to treat the defect caused by the polymorphism or to subject the individual to behavioral modification protocols to help ameliorate the symptoms.

Another aspect of the present invention provides a method a method for determining the likelihood of a subject responding favorably to a particular drug in the treatment of a neurological, psychiatric or psychological condition, phenotype or state said method comprising obtaining or extracting a DNA sample from cells of said individual and screening for or otherwise detecting the presence of from about one to about 100 polymorphisms in one or more genes listed in Table 2 including their 5′ or 3′ terminal region, promoter, intron or exons which with a statistical significant association with a particular neurological, psychiatric or psychological condition, phenotype or state wherein the presence of the polymorphism profile is indicative of the likelihood of the drug being effective.

Using the compositions of the present invention, gene therapy may be recommended when a particular polymorphism conferring, for example, a disease condition or a propensity for development of neurological, psychiatric or psychological condition, phenotype or state is identified in an embryo. Genetically modified stem cells may then be used to alter the genotype of the developing cells. Where an embryo has developed into a fetus or for post-natal subjects, localized gene therapy may still be accomplished. Alternatively, a compound may be identified which effectively masks a particular undesired polymorphic variant or which influences the expression of a more desired phenotype. For example, one polymorphic variant of a receptor may result in an instability of the mRNA transition product.

Accordingly, the present invention also provides genetic test kits which allow the rapid screening of a polymorphism or polymorphisms within a test sample or multiple test samples. The kits of the present invention comprise one or more sets of primers, as described herein, which are specific for the amplification of a genetic region of interest. In addition, the genetic testing kits of the present invention provide a PCR mix, comprising MgCl₂. In a preferred aspect, the MgCl₂ is provided at a concentration of 2.5 mM. Additionally, the genetic test kits of the present invention provide instructions for using the primers of the present invention to obtain the desired duplexes, as well as instructions as to the analysis of the duplexes using d-HPLC. The test kits may also contain instructions for use.

In essence, the identification of a panel of polymorphisms in one or more genes as listed in Table 2 or 3 (or a subset) allows a clinician to confirm behavioral characteristics or provide a diagnosis of schizophrenia. Again, reference to the panel in Table 2 or 3 includes all genes listed in Tables 2 and/or 3 or combination of two or more genes.

Therapeutic kits are also contemplated by the present invention. For example, the kit may comprise a diagnostic or polymorphism detection component and a selection of therapeutics, the choice of use of which is dependent on the outcome of the diagnostic assay.

The present invention is further described with reference to the following non-limiting Examples.

### Example 1

### Selection of Patients

The clinical data pertaining to schizophrenia patients are comprehensive and allow for the appraisal of specific phenotypic groups based on symptoms, history or response to medication. Patients were being treated at the Fortitude Valley Community Mental Health Centre, the Royal Brisbane Mental Health Unit and the Park Psychiatric Hospital each in Brisbane, Australia. Inclusion criteria were being between 18 and 65 years of age and having a DSM IV diagnosis of schizophrenia. In this particular study potential participants were excluded if they had Schizoaffective Disorder, Bipolar Disorder, Dementia, Organic Brain Syndrome or Major Depressive Disorder with Delusions. The clinical test battery includes measures of symptom type and severity (e.g. Positive and Negative Symptoms Scale (PANSS) or Positive and Negative Symptoms Test (PANDT) or Positive and Negative Symptoms Scale Total (PANSST)), medication adverse effects (e.g. Barnes Akathisia Scale) and neuropsychological status (e.g. Reitan's Trail Making Test). All patients meet DSM IV (Diagnostic and Statistical Manual of Mental Disorders, 4^{th} Ed.) criteria for the diagnosis of schizophrenia. The clinical diagnoses of the patients that are being used for the study are of high quality regarding the accuracy of specific disease diagnosis. Patients are psychiatrically assessed by at least three independent psychiatrists and all must confirm the same diagnosis for patients to be included. These patients must have had a “stable and severe” diagnosis and have previously been in inpatient care and have displayed symptoms for at least five years and be currently undergoing antipsychotic drug treatment. Patients must have no previous diagnosis of any other disorders such as schizoaffective disorder, bipolar disorder, depression etc. It is also necessary for inclusion into the patient database that there be no previous history of drug dependence.

A parallel comprehensive data set to that obtained from those with schizophrenia exists to enable a finely grained understanding of the alcohol dependence phenotypes. The battery includes alcohol dependence symptoms (e.g. the Alcohol Dependence Scale), affective state (e.g. Beck Depression Inventory, Spielbergers State-Trait Anxiety Inventory), craving (Borg scale) and neuropsychological status (e.g. Reitan's Trail Making Test).

A 10 mL blood sample was drawn from each subject for DNA extraction.

### Example 2

### Genotyping

The schizophrenia patient database is screened for functional polymorphisms in genes that are involved in the dopamine pathway and associated receptor genes. Further work has been performed to comprehensively screen the remaining SNPs of the dopamine D2 receptor (DRD2) gene, in addition further genes include catechol-O-methyl transferase (COMT), protein kinase B (AKT1), dopamine associated transporter (DAT), ankyrin repeat and protein kinase domain-containing protein 1 (ANKK1), gamma-aminobutyric acid A receptor alpha 1 (GABRA1), glutamate receptor metabotropic 3 (GRM3), serotonin receptor 2A (HTR2A), karyopherin alpha 3 (KPNA3), proline dehydrogenase 1 (PRODH), regulator of G-protein signalling 4 (RGS4), disrupted in schizophrenia (DISC1), and dysbindin (DTNBP1) will be targeted.

The selected SNPs have been processed using the schizophrenia (n=160) and control (n=250) samples that are available. Sample numbers are sufficient to detect alleles accounting for 1-5% of genetic variation with a power of between 74% and 99%.

### Example 3

### Methods

SEQUENOM [Trade Mark] has protocols optimized for multiplexing the homogeneous MassEXTEND (hME) assay. The hME assay is a simple and robust method for the analysis of single nucleotide polymorphisms (SNPs). The speed and accuracy of matrix-assisted laser desorption/ionization time-of-flight mass spectrometry (MALDI-TOF MS) offers a solution for high-throughput genotyping. The hME assay is based on the annealing of an oligonucleotide primer (hME primer) adjacent to the SNP of interest. The addition of a DNA polymerase along with a mixture of terminator nucleotides allows extension of the hME primer through the polymorphic site and generates allele-specific extension products, each having a unique molecular mass. The resultant masses of the extension products are then analyzed by MALDI-TOF MS and a genotype is assigned in real time. Performing multiple PCR and hME reactions in a single well (multiplexing) is a way to further increase the throughput and reduce cost per genotype. SEQUENOM has optimized individual reagent concentrations and thermal cycling conditions to multiplex PCR and hME reactions for best MALDI-TOF-MS-based genotyping results.

A list of primers used is shown in Table 7.

TABLE 7
Primer list of SNPs that are associated with Schizophrenia.

| | | | | | | | | | | |
|---|---|---|---|---|---|---|---|---|---|---|
| | | | | AMP_{—} | UP_{—} | MP_{—} | Tm | | P | UEP_{—} |
| GENE | SNP_ID | 2nd-PCRP | 1st-PCRP | LEN | CONF | CONF | (NN) | PcGC | WARN | DIR |

| | | | | | | | | | | |
|---|---|---|---|---|---|---|---|---|---|---|
| DRD2 | rs6277 | ACGTTGGATGAGCCACCACCAGCTGA | ACGTTGGATGATTCTTCTCTGGTTT | 104 | 91.9 | 67.3 | 49.1 | 62.5 | d | F |
| | | CTCT | GGCGG | | | | | | | |
| DTNBP1 | RS9370822 | ACGTTGGATGAGCTGACTCACACAGT | ACGTTGGATGCGGTTTTGAAAGGAA | 101 | 99.9 | 67.3 | 46.2 | 38.9 | dg | R |
| | | GATG | CTGCC | | | | | | | |
| DTNBP1 | RS1997679 | ACGTTGGATGATAGTTCGAAGCCACT | ACGTTGGATGGCCTAAATACCTCAA | 101 | 98.6 | 78.6 | 49.5 | 34.8 | sd | F |
| | | CTGG | TCCTG | | | | | | | |
| GABRA1 | RS4263535 | ACGTTGGATGTGTAAGAAAGTAGCAG | ACGTTGGATGCTGGATTCATTCTTG | 88 | 88,9 | 68.4 | 60.6 | 53.8 | h | R |
| | | CCCC | TCC | | | | | | | |
| DAT | RS40184 | ACGTTGGATGCACAGTCTCGCGGCTT | ACGTTGGATGAACACACCCTTGACA | 114 | 95.3 | 67.3 | 47 | 23.1 | D | F |
| | | TTTA | GGTGC | | | | | | | |
| DAT | RS2975292 | ACGTTGGATGTGACAGGTGCAGTGAG | ACGTTGGATGTCAGTGCTGCAGGCA | 117 | 93.6 | 91.1 | 48.4 | 50 | D | F |
| | | AGAG | CACTT | | | | | | | |
| DAT | RS13161905 | ACGTTGGATGACTGCCATCATGCAGC | ACGTTGGATGATCCTCCCTTGATCA | 103 | 94.5 | 67.3 | 53.9 | 68.8 | d | R |
| | | AGG | GGGTC | | | | | | | |
| DAT | RS11133767 | ACGTTGGATGTGAACGCTGAACGTGC | ACGTTGGATGTGAAGATGCTGCCTG | 113 | 96.5 | 62.1 | 49.6 | 62.5 | D | R |
| | | CTTC | CTCTG | | | | | | | |
| COMT | RS4680 | ACGTTGGATGTTTTCCAGGTCTGACA | ACGTTGGATGACCCAGCGGATGGTG | 94 | 95.9 | 67.3 | 45.8 | 50 | | R |
| | | ACGG | GATTT | | | | | | | |
| COMT | RS165774 | ACGTTGGATGTTCCAATCCGTGTCCA | ACGTTGGATGGAAACTGGACACTGC | 120 | 90.8 | 67.3 | 48 | 62.5 | D | R |
| | | GGGT | TGTTA | | | | | | | |
| DAT | R54975646 | ACGTTGGATGATTGGCAGTCTTGATG | ACGTTGGATGTAGGGAGCCCATGCA | 109 | 99.7 | 62.1 | 58.1 | 56.5 | Dh | R |
| | | GCTC | AATAG | | | | | | | |
| RGS4 | RS2842030 | ACGTTGGATGCCGTACAAAAATACAA | ACGTTGGATGTAACTCTTTGAGGAG | 120 | 87.4 | 85.2 | 46.8 | 35 | D | F |
| | | GAGTG | AGAGC | | | | | | | |
| KPNA3 | RS9562919 | ACGTTGGATGCGAACCATATGACTTT | ACGTTGGATGGAAGTTTTGCTACTA | 117 | 88 | 85.2 | 47.6 | 35 | D | F |
| | | TGTG | GTGAG | | | | | | | |
| AKT1 | RS3001371 | ACGTTGGATGGCCAGTTTTTATCTCC | ACGTTGGATGGTTTGGAAACTGGCC | 90 | 98.2 | 72.2 | 55.1 | 68.8 | D | F |
| | | AGCC | CAGTC | | | | | | | |
| HTR2A | RS2770297 | ACGTTGGATGCATAACTAATTGTAGT | ACGTTGGATGGAGTTTCAGGCTAGC | 120 | 76.2 | 83.2 | 46.3 | 30.4 | H | R |
| | | CCA | TTCTG | | | | | | | |
| PRODH | RS5747933 | ACGTTGGATGACCTGCAGCACCGCCA | ACGTTGGATGTTCACCAAATGGCTG | 99 | 80.8 | 74.6 | 53.3 | 68.8 | s | F |
| | | CCT | TGGAG | | | | | | | |
| PRODH | RS2238733 | ACGTTGGATGGAAAGCACAAAGCAGC | ACGTTGGATGTGCTCCAGCTCTGGG | 113 | 93.7 | 72.2 | 50.5 | 58.8 | d | F |
| | | TGAG | AAGTG | | | | | | | |
| PRODH | RS2870984 | ACGTTGGATGCAGAGATCGGCTATGA | ACGTTGGATGACCTGTGGTACATGG | 86 | 96.4 | 74 | 56.4 | 61.1 | d | R |
| | | GGAC | CGTTG | | | | | | | |
| ANKK1 | RS1800497 | ACGTTGGATGCAACACAGCCATCCTC | ACGTTGGATGTGTGCAGCTCACTCC | 93 | 95.9 | 72.2 | 60.2 | 56 | d | P |
| | | AAAG | ATCCT | | | | | | | |
| DISC1 | RS6675281 | ACGTTGGATGACAACGTGCTGTAGGA | ACGTTGGATGGCCCTTCTCTCTCTG | 103 | 97.4 | 67.3 | 48.5 | 52.9 | | F |
| | | AACC | ATGTT | | | | | | | |
| GRM3 | P62214653 | ACGTTGGATGAGTGTGATGACCATAA | ACGTTGGATGCCTCAAATGTATTCG | 99 | 93 | 72.2 | 56.7 | 55 | Sd | R |
| | | ACCC | GAATG | | | | | | | |
| RGS4 | RS10759 | ACGTTGGATGACAACTGAAAAACACA | ACGTTGGATGCTCAGAGACTGCTGT | 118 | 86.8 | 72.2 | 46.8 | 22.2 | D | F |
| | | CTC | CTTAC | | | | | | | |

| | | | | | | | | |
|---|---|---|---|---|---|---|---|---|
| | | | EXT1_{—} | EXT1_{—} | | EXT2_{—} | EXT2_{—} | |
| GENE | SNP_ID | UEP_SEQ | CALL | MASS | EXT1_SEQ | CALL | MASS | EXT2_SEQ |

| | | | | | | | | |
|---|---|---|---|---|---|---|---|---|
| DRD2 | rs6277 | tGTCTCCACAGCACTCC | C | 5313.5 | tGTCTCCACAGCACTCCC | T | 5393.4 | tGTCTCCACAGCACTCCT |
| DTNBP1 | RS9370822 | TGGGGATGTAAAATGGAA | C | 5938.9 | TGGGGATGTAAAATGGAAG | A | 5978.8 | TGGGGATGTAAAATGGAAT |
| DTNBP1 | RS1997679 | GACTTTTACCTTTTGAGCT | C | 7257.8 | GACTTTTACCTTTTGAGCT | T | 7337.7 | GACTTTTACCTTTTGAGCT |
| | | GTTA | | | GTTAC | | | GTTAT |
| GABRA1 | RS4263535 | CCCCCACACCTTGCCACCA | G | 8045.3 | CCCCCACACCTTGCCACCA | A | 8125.2 | CCCCCACACCTTGCCACCA |
| | | AATAAAG | | | AATAAAGC | | | AATAAAGT |
| DAT | RS40184 | AAAATCAAGTAATGATTGA | A | 8327.5 | AAAATCAAGTAATGATTGA | G | 8343.5 | AAAATCAAGTAATGATTGA |
| | | TTTGTAG | | | TTTGTAGA | | | TTTGTAGG |
| DAT | RS2975292 | ACATGGCACCTATGAG | C | 5137.4 | ACATGGCACCTATGAGC | G | 5177.4 | ACATGGCACCTATGAGG |
| DAT | R513161905 | GGGCGCACATGGGATG | T | 5258.5 | GGGCGCACATGGGATGA | C | 5274.5 | GGGCGCACATGGGATGG |
| DAT | R511133767 | CCTTCCTTCCACTGCC | G | 4951.2 | CCTTCCTTCCACTGCCC | A | 5031.2 | CCTTCCTTCCACTGCCT |
| COMT | RS4680 | ttCACACCTTGTCCTTCA | G | 5607.7 | ttCACACCTTGTCCTTCAC | A | 5687.6 | ttCACACCTTGTCCTTCAT |
| COMT | RS165774 | CCTCGTGCTCCTAGTC | G | 5031.3 | CCTCGTGCTCCTAGTCC | A | 5111.2 | CCTCGTGCTCCTAGTCT |
| DAT | RS4975646 | GGGTGTTCCTGGGTAACCC | G | 7342.8 | GGGTGTTCCTGGGTAACCC | A | 7422.7 | GGGTGTTCCTGGGTAACCC |
| | | TAGA | | | TAGAC | | | TAGAT |
| RGS4 | RS2842030 | AAAATACAAGAGTGTCAGG | G | 6494.3 | AAAATACAAGAGTGTCAGG | T | 6534.2 | AAAATACAAGAGTGTCAGG |
| | | A | | | AG | | | AT |
| KPNA3 | RS9562919 | gacaATATGACTTTTGTGA | A | 7629 | gacaATATGACTTTTGTGA | T | 7684.9 | gacaATATGACTTTTGTGA |
| | | CTGCT | | | CTGCTA | | | CTGCTT |
| AKT1 | RS3001371 | CCCCACCCAAACCCCA | C | 4932.3 | CCCCACCCAAACCCCAC | T | 5012.2 | CCCCACCCAAACCCCAT |
| HTR2A | RS2770297 | ACTAATTGTAGTCCAATTT | T | 7292.8 | ACTAATTGTAGTCCAATTT | C | 7308.8 | ACTAATTGTAGTCCAATTT |
| | | AGAC | | | AGACA | | | AGACG |
| PRODH | RS5747933 | CGCCACCTCCAGCTTG | G | 5065.3 | CGCCACCTCCAGCTTGG | T | 5105.2 | CGCCACCTCCAGCTTGT |
| PRODH | RS2238733 | GTGAGGACAGGAGGGAA | C | 5620.7 | GTGAGGACAGGAGGGAAC | A | 5644.7 | GTGAGGACAGGAGGGAAA |
| PRODH | RS2870984 | cAGGACCCCATCAACCCCA | G | 5918.9 | cAGGACCCCATCAACCCCA | A | 5998.8 | CAGGACCCCATCAACCCCA |
| | | | | | C | | | T |
| ANKK1 | RS1800497 | cCACAGCCATCCTCAAAGT | T | 8147.3 | CCACAGCCATCCTCAAAGT | C | 8163.3 | CCACAGCCATCCTCAAAGT |
| | | GCTGGTC | | | GCTGGTCA | | | GCTGGTCG |
| DISC1 | RS6675281 | TCTGGACGGCTAAAGAC | C | 5466.6 | TCTGGACGGCTAAAGACC | T | 5546.5 | TCTGGACGGCTAAAGACT |
| GRM3 | RS2214653 | tACCATAAACCCTGAGCCC | G | 6551.3 | tACCATAAACCCTGAGCCC | A | 6631.2 | tACCATAAACCCTGAGCCC |
| | | CA | | | CAC | | | CAT |
| RGS4 | RS10759 | ACATAAAATAATTTACTTC | C | 8447.6 | ACATAAAATAATTTACTTC | A | 8471.6 | ACATAAAATAATTTACTTC |
| | | TCATTCAG | | | TCATTCAGC | | | TCATTCAGA |

### Example 4

### Procedure Used to Identify Panel of SNPs Associated with Schizophrenia

In two stages, a total of 172 SNPs were analyzed for association with schizophrenia. This followed identification of genes and a total of 273 SNPs that had been suggested to be involved in the pathogenesis of schizophrenia from the literature. These SNPs were then genotyped as described in Example 3. Some SNPs failed to amplify, some were monomorphic in our populations, while a small number, upon scrutiny, gave results that were inconsistent with expectation. These SNPs were removed from further analysis.

The remaining SNPs were analysed for association using Chi Square tests as described. However, it was expected that some SNPs found to be associated with schizophrenia would also be associated with each other. That is, once the genotype of one SNP was known, a knowledge of the genotype of a second SNP would not significantly add to the ability to diagnose schizophrenia, even though the second SNP was also associated with schizophrenia.

Thus, the intention was to identify the smallest set of SNPs that would give the greatest discriminating ability between control and schizophrenic individuals. To achieve this, two different methods were used: Binary Logistic Regression and Discriminant Function Analysis. Both methods build a model in which SNPs are sequentially added based on the extent and significance of association with schizophrenia. The most associated SNP is added first, than the most associated SNP independent of the first added SNP, then the most associated SNP independent of the first two added SNPs, and so on. No more SNPs are added to the list once the addition of new SNPs does not significantly improve the discriminating ability of the model.

One problem with the use of these methods is that missing data render calculations impossible. Obviously, when investigating 410 individuals for 172 SNPs there were many instances of missing data. To overcome the problem of missing data the method of “imputation of missing values” was used (Donders et al, J Clin Epidemiol 59(10):1087-1091, 2006; Burd et al, J Trauma 60(4):792-801, 2006; van der Heijden et al, J Clin Epidemiol 59(10):1102-1109, 2006). Specifically, for each SNP, the distribution of genotype frequencies was calculated for controls and schizophrenics. These frequencies were then used to generate a total of 260 “virtual” genotypes with identical genotype-frequency distributions to the original control and schizophrenic populations. Then for each missing genotype a virtual genotype was imputed to the data set that was randomly selected from the 260 virtual genotypes of the relevant SNP and schizophrenia/control designation.

Once the imputations had been done binary logistic regression and discriminant function analysis could be performed and a panel of SNPs generated that best predicted the schizophrenia/control status of individuals. To avoid any stochastic biases due to the imputation method, the process was repeated 10 times and the final panel of SNPs was selected on the basis of consistency of inclusion in the 10 generated models for each of the binary logistic regression and discriminant function analysis methods.

Table 2 lists the genes of interest and or a combination of SNPs listed in Table 3 lists the panel so far selected to best test for schizophrenia.

### Example 5

### Linkage Disequilibrium Analysis

Linkage disequilibrium (LD) describes a situation in which some combinations of alleles or genetic markers occur more or less frequently in a population than would be expected from a random formation of haplotypes. LD is an important factor to examining association for disease susceptibility loci. This data describes the SNPs that are associated with schizophrenia that display LD. The identification of SNPs displaying LD explains the inclusion/exclusion of individual SNPs that are included in the discriminant analysis and logistic regression models.

The JLIN software package was used to generate LD values (Carter et al, BMC Bioinformatics 7(1):60, 2006).

Results of linkage disequilibrium analysis are shown in Table 8.

TABLE 8
List 1 - Linkage Disequilibrium Analysis

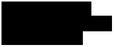

| | |
|---|---|
| 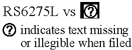 | |
| ****************** | |
| D = 0.16546227946343617 | |
| Dmin = −0.15124594996299773 | |
| Dmax = 0.16546227946343617 | |
| D′ = 1.0 | |
| r\hat{ }2 = 0.5070705723161366 | |
| OddsRatio = Infinity | |
| Pexcess = 7.558668960058087E−4 | |
| d = 1.706024285426338E−7 | |
| Q = NaN | |
| Haplotypes | |
| TC = 0.3167082294264339 | |
| TT = 0.0 | |
| CC = 0.16084788029925187 | |
| CT = 0.5224438902743143 | |
| Alleles | |
| RS6275L | |
| Allele: C | count = 556 |
| freq = 0.6847290640394089 | |
| Allele: T | count = 256 |
| freq = 0.31527093596059114 | |
| rs6277L | |
| Allele: C | count = 386 |
| freq = 0.47890818858560796 | |
| Allele: T | count = 420 |
| freq = 0.5210918114143921 | |
| Genotypes | |
| RS6275L | |
| Genotype: CC | count = 193 |
| freq = 0.4753694581280788 | |
| Genotype: TT | count = 43 |
| freq = 0.10591133004926108 | |
| Genotype: TC | count = 170 |
| freq = 0.4187192118226601 | |
| rs6277L | |
| Genotype: CC | count = 90 |
| freq = 0.22332506203473945 | |
| Genotype: TT | count = 107 |
| freq = 0.2655086848635236 | |
| Genotype: CT | count = 206 |
| freq = 0.511166253101737 | |
| 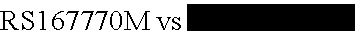 | |
| *********************** | |
| D = 0.13035263216039444 | |
| Dmin = 0.06296756953124519 | |
| Dmax = 0.1615981872925762 | |
| D′= 0.8066466236059248 | |
| r\hat{ }2 = 0.4835982855847828 | |
| OddsRatio = 48.91031170700547 | |
| Pexcess = 0.0037253234885804487 | |
| d = 1.5020383391065723E−7 | |
| Q = 0.9599281204304866 | |
| Haplotypes | |
| GG = 0.0870768206408666 | |
| GC = 0.1933202016916396 | |
| AG = 0.6883574225353122 | |
| AC = 0.03124555513218174 | |
| Alleles | |
| RS167770M | |
| Allele: A | count = 583 |
| freq = 0.7179802955665024 | |
| Allele: G | count = 229 |
| freq = 0.28201970443349755 | |
| RS1800828M | |
| Allele: C | count = 181 |
| freq = 0.2234567901234568 | |
| Allele: G | count = 629 |
| freq = 0.7765432098765432 | |
| Genotypes | |
| RS167770M | |
| Genotype: GG | count = 30 |
| freq = 0.07389162561576355 | |
| Genotype: AA | count = 207 |
| freq = 0.5098522167487685 | |
| Genotype: GA | count = 169 |
| freq = 0.41625615763546797 | |
| RS1800828M | |
| Genotype: CC | count = 23 |
| freq = 0.056790123456790124 | |
| Genotype: GG | count = 247 |
| freq = 0.6098765432098765 | |
| Genotype: GC | count = 135 |
| freq = 0.3333333333333333 | |
| 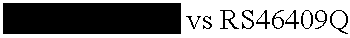 | |
| ************************ | |
| D = 0.1862126714457965 | |
| Dmin = −0.20673490925807222 | |
| Dmax = 0.18766712636533742 | |
| D′ = 0.9922498151503131 | |
| r\hat{ }2 = 0.5820645912310127 | |
| OddsRatio = 976.6429348392656 | |
| Pexcess = 0.0011502106787363788 | |
| d = 2.250347788729827E−7 | |
| Q = 0.9979542633320121 | |
| Haplotypes | |
| TC = 0.001454454919540908 | |
| TT = 0.39294758070386876 | |
| CC = 0.4743725170906372 | |
| CT = 0.13122544728595312 | |
| Alleles | |
| RS13161905Q | |
| Allele: C | count = 486 |
| freq = 0.6029776674937966 | |
| Allele: T | count = 320 |
| freq = 0.3970223325062035 | |
| RS464049Q | |
| Allele: C | count = 380 |
| freq = 0.47738693467336685 | |
| Allele: T | count = 416 |
| freq = 0.5226130653266332 | |
| Genotypes | |
| RS13161905Q | |
| Genotype: CC | count = 151 |
| freq = 0.3746898263027295 | |
| Genotype: TT | count = 68 |
| freq = 0.1687344913151365 | |
| Genotype: TC | count = 184 |
| freq = 0.456575682382134 | |
| RS464049Q | |
| Genotype: CC | count = 95 |
| freq = 0.23869346733668342 | |
| Genotype: TT | count = 113 |
| freq = 0.28391959798994976 | |
| Genotype: CT | count = 190 |
| freq = 0.47738693467336685 | |
| 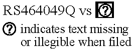 | |
| *********************** | |
| D = 0.15743316198456064 | |
| Dmin = 0.1757593157751144 | |
| Dmax = 0.16051272452715265 | |
| D′ = 0.9808142154980924 | |
| r\hat{ }2 = 0.44510785938604214 | |
| OddsRatio = 270.8046528761808 | |
| Pexcess = 0.0017696597317208684 | |
| d = 1.7614509727492138E−7 | |
| Q = 0.9926417742344128 | |
| Haplotypes | |
| CG = 0.47425041226849113 | |
| CA = 0.0030795625425919806 | |
| TG = 0.18947754742924183 | |
| TA = 0.33319247775967503 | |
| Alleles | |
| RS464049Q | |
| Allele: C | count = 380 |
| freq = 0.47738693467336685 | |
| Allele: T | count = 416 |
| freq = 0.5226130653266332 | |
| RS4975646Q | |
| Allele: A | count = 277 |
| freq = 0.3402948402948403 | |
| Allele: G | count = 537 |
| freq = 0.6597051597051597 | |
| Genotypes | |
| RS464049Q | |
| Genotype: CC | count = 95 |
| freq = 0.23869346733668342 | |
| Genotype: TT | count = 113 |
| freq = 0.28391959798994976 | |
| Genotype: CT | count = 190 |
| freq = 0.47738693467336685 | |
| RS4975646Q | |
| Genotype: GG | count = 179 |
| freq = 0.4398034398034398 | |
| Genotype: AA | count = 49 |
| freq = 0.12039312039312039 | |
| Genotype: GA | count = 179 |
| freq = 0.4398034398034398 | |
| 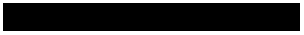 | |
| ******************** | |
| D = 0.15238125 | |
| Dmin = −0.17011874999999999 | |
| Dmax = 0.15238125 | |
| D′ = 1.0 | |
| r\hat{ }2 = 0.42638288942131125 | |
| OddsRatio = Infinity | |
| Pexcess = 0.0011196682464454976 | |
| d = 1.4556561944609005E−7 | |
| Q = NaN | |
| Haplotypes | |
| GG = 0.4725 | |
| GA = 0.205 | |
| AG = 0.0 | |
| AA = 0.3225 | |
| Alleles | |
| RS165774T | |
| Allele: A | count = 259 |
| freq = 0.32054455445544555 | |
| Allele: G | count = 549 |
| freq = 0.6794554455445545 | |
| RS4680T | |
| Allele: A | count = 429 |
| freq = 0.5296296296296297 | |
| Allele: G | count = 381 |
| freq = 0.4703703703703704 | |
| Genotypes | |
| RS165774T | |
| Genotype: GG | count = 198 |
| freq = 0.4900990099009901 | |
| Genotype: AA | count = 53 |
| freq = 0.1311881188118812 | |
| Genotype: GA | count = 153 |
| freq = 0.3787128712871287 | |
| RS4680T | |
| Genotype: GG | count = 98 |
| freq = 0.2419753086419753 | |
| Genotype: AA | count = 122 |
| freq = 0.3012345679012346 | |
| Genotype: GA | count = 185 |
| freq = 0.4567901234567901 | |
| 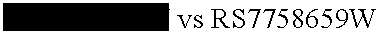 | |
| ************************ | |
| D = 0.14627120212695627 | |
| Dmin = 0.06994534590179262 | |
| Dmax = 0.15745990191161846 | |
| D′ = 0.9289425456968571 | |
| r\hat{ }2 = 0.5717913633420962 | |
| OddsRatio = 144.08887792251366 | |
| Pexcess = 0.00433655943367277 | |
| d = 2.5877194692386185E−7 | |
| Q = 0.9862153458718722 | |
| Haplotypes | |
| CC = 0.681231125288224 | |
| CT = 0.011188699784662182 | |
| TC = 0.0913636268983548 | |
| TT = 0.2162165480287489 | |
| Alleles | |
| RS1997679W | |
| Allele: C | count = 552 |
| freq = 0.6781326781326781 | |
| Allele: T | count = 262 |
| freq = 0.32186732186732187 | |
| RS7758659W | |
| Allele: C | count = 532 |
| freq = 0.7732558139534884 | |
| Allele: T | count = 156 |
| freq = 0.22674418604651161 | |
| Genotypes | |
| RS1997679W | |
| Genotype: CC | count = 188 |
| freq = 0.4619164619164619 | |
| Genotype: TT | count = 43 |
| freq = 0.10565110565110565 | |
| Genotype: CT | count = 176 |
| freq = 0.43243243243243246 | |
| RS7758659W | |
| Genotype: CC | count = 207 |
| freq = 0.6017441860465116 | |
| Genotype: TT | count = 19 |
| freq = 0.055232558139534885 | |
| Genotype: CT | count = 118 |
| freq = 0.3430232558139535 | |
| 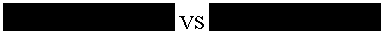 | |
| ************************ | |
| D = 0.18764617964580232 | |
| Dmin = 0.15702611760935747 | |
| Dmax = 0.19740426213747797 | |
| D′ = 0.9505680252998802 | |
| r\hat{ }2 = 0.623647076263382 | |
| OddsRatio = 196.49319951396126 | |
| Pexcess = 6.723885863845475E−4 | |
| d = 2.4657625165031323E−7 | |
| Q = 0.9898730690225178 | |
| Haplotypes | |
| CC = 0.009758082491675635 | |
| CA = 0.54720394282478 | |
| TC = 0.3446722972551598 | |
| TA = 0.0983656774283845 | |
| Alleles | |
| RS4236167W | |
| Allele: C | count = 446 |
| freq = 0.554726368159204 | |
| Allele: T | count = 358 |
| freq = 0.44527363184079605 | |
| RS9370822W | |
| Allele: A | count = 517 |
| freq = 0.64625 | |
| Allele: C | count = 283 |
| freq = 0.35375 | |
| Genotypes | |
| RS4236167W | |
| Genotype: CC | count = 128 |
| freq = 0.31840796019900497 | |
| Genotype: TT | count = 84 |
| freq = 0.208955223880597 | |
| Genotype: CT | count = 190 |
| freq = 0.472636815920398 | |
| RS9370822W | |
| Genotype: CC | count = 56 |
| freq = 0.14 | |
| Genotype: CA | count = 171 |
| freq = 0.4275 | |
| Genotype: AA | count = 173 |
| freq = 0.4325 | |
| 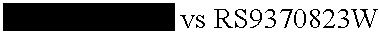 | |
| ************************ | |
| D = 0.1474381878237005 | |
| Dmin = 0.12543765275091573 | |
| Dmax = 0.15635785847102945 | |
| D′ = 0.9429534867351638 | |
| r\hat{ }2 = 0.43486884212157423 | |
| OddsRatio = 96.95628012057838 | |
| Pexcess = 4.6885663031561375E−4 | |
| d = 1.6698945449803946E−7 | |
| Q = 0.9795827281564988 | |
| Haplotypes | |
| CG = 0.008919670647328948 | |
| CA = 0.5459431722454392 | |
| TG = 0.2728758405746162 | |
| TA = 0.1722613165326157 | |
| Alleles | |
| RS4236167W | |
| Allele: C | count = 446 |
| freq = 0.554726368159204 | |
| Allele: T | count = 358 |
| freq = 0.44527363184079605 | |
| RS9370823W | |
| Allele: A | count = 587 |
| freq = 0.7211302211302212 | |
| Allele: G | count = 227 |
| freq = 0.2788697788697789 | |
| Genotypes | |
| RS4236167W | |
| Genotype: CC | count = 128 |
| freq = 0.31840796019900497 | |
| Genotype: TT | count = 84 |
| freq = 0.208955223880597 | |
| Genotype: CT | count = 190 |
| freq = 0.472636815920398 | |
| RS9370823W | |
| Genotype: GG | count = 32 |
| freq = 0.07862407862407862 | |
| Genotype: AA | count = 212 |
| freq = 0.5208845208845209 | |
| Genotype: GA | count = 163 |
| freq = 0.4004914004914005 | |
| 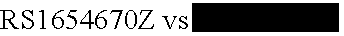 | |
| *********************** | |
| D = 0.11896416288014353 | |
| Dmin = −0.033604568258972664 | |
| Dmax = 0.1415872475978048 | |
| D′ = 0.840218062703466 | |
| r\hat{ }2 = 0.6317870197649801 | |
| OddsRatio = 134.98509098129603 | |
| Pexcess = 0.0056915433941564005 | |
| d = 1.6015924913453652E−7 | |
| Q = 0.9852925053359336 | |
| Haplotypes | |
| GG = 0.785561058504845 | |
| GA = 0.02262308471766128 | |
| CG = 0.03924712563837739 | |
| CA = 0.1525687311391162 | |
| Alleles | |
| RS1654670Z | |
| Allele: C | count = 159 |
| freq = 0.1962962962962963 | |
| Allele: G | count = 651 |
| freq = 0.8037037037037037 | |
| RS276713Z | |
| Allele: A | count = 137 |
| freq = 0.17341772151898735 | |
| Allele: G | count = 653 |
| freq = 0.8265822784810126 | |
| Genotypes | |
| RS1654670Z | |
| Genotype: CC | count = 15 |
| freq = 0.037037037037037035 | |
| Genotype: GG | count = 261 |
| freq = 0.6444444444444445 | |
| Genotype: GC | count = 129 |
| freq = 0.31851851851851853 | |
| RS276713Z | |
| Genotype: GG | count = 271 |
| freq = 0.6860759493670886 | |
| Genotype: AA | count = 13 |
| freq = 0.03291139240506329 | |
| Genotype: GA | count = 111 |
| freq = 0.2810126582278481 | |
| 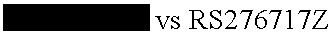 | |
| ********************** | |
| D = 0.1394385569660234 | |
| Dmin = 0.033199596131363585 | |
| Dmax = 0.14205813582739926 | |
| D′ = 0.9815598110863664 | |
| r\hat{ }2 = 0.8760437222515121 | |
| OddsRatio = 3170.389535916304 | |
| Pexcess = 0.005494507413310144 | |
| d = 2.102799083824552E−7 | |
| Q = 0.99936936160716 | |
| Haplotypes | |
| GC = 0.8079474314479024 | |
| GA = 0.016794836593334642 | |
| AC = 0.00261957886137588 | |
| AA = 0.172638153097387 | |
| Alleles | |
| RS276713Z | |
| Allele: A | count = 137 |
| freq = 0.17341772151898735 | |
| Allele: G | count = 653 |
| freq = 0.8265822784810126 | |
| RS276717Z | |
| Allele: A | count = 154 |
| freq = 0.19201995012468828 | |
| Allele: C | count = 648 |
| freq = 0.8079800498753117 | |
| Genotypes | |
| RS276713Z | |
| Genotype: GG | count = 271 |
| freq = 0.6860759493670886 | |
| Genotype: AA | count = 13 |
| freq = 0.03291139240506329 | |
| Genotype: GA | count = 111 |
| freq = 0.2810126582278481 | |
| RS276717Z | |
| Genotype: CC | count = 265 |
| freq = 0.06608478802992519 | |
| Genotype: AA | count = 18 |
| freq = 0.04488778054862843 | |
| Genotype: CA | count = 118 |
| freq = 0.2942643391521197 | |

### Example 6

### SNP Correlations with Clinical and Phenotypic Schizophrenia Patient Data

These data describe the correlation between the number of each allele in a particular individual and the phenotype of interest.

[1] Early Detection—Reported Age of Onset of schizophrenia and Family History Onset Age and Family History

### [2] Assessment of Clinical Features of Schizophrenia Symptoms and Markers of Severity

Suicide attempts, negative symptoms and number of admissions

### [3] Assessment of Comorbidity and Health Problems (Mental and Physical)

Number of cigarettes/day, grams/hour alcohol, mg/day cigarettes, pulmonary function, gambling history, Alcohol Use Disorders Identification Test (AUDIT), Trail Making Test A (TMTA) and Trail Making Test B (TMTB), impulsivity, General Health Questionnaire 1 (GHQ1), GHQ2, GHQ3, GHQ4 and GHQ Total (GHQT).

### [4] Medication Response (Adverse Medication Effects)

Glucose, prolactin (IU/I), and type of antipsychotic drugs (Risperidone, Olanzapine, Clozapine, Seroquel and Typical).

The following SNPs were found to be significant

### [1] Early Detection—Reported Age of Onset of Schizophrenia and Family History

Rs6277 (DRD2) with onset age (p=0.013). The CC (0.037 Tukey and 0.041 Bonferroni) and CT (0.013 Tukey and 0.014 Bonferroni) genotypes were associated with late onset age. An ANOVA was performed on the following data, comparing the mean of phenotypic data with genotype.

Rs4263535 (GABRA1) with onset age (p=0.046). Genotype GG has later onset age compared to AA (not significant p=0.181 Tukey). An ANOVA was performed on the following data, comparing the mean of phenotypic data with genotype.

Rs9562919 (KPNA3) with family history (p=0.013). A Pearson chi-squared test was performed.

Rs40184 (DAT) with family history (p=0.009). A Pearson chi-squared test was performed.

### [2] Assessment of Clinical Features of Schizophrenia Symptoms and Markers of Severity

Rs4263535 (GABRA1) with number of admissions (p=0.025)

### GG=43.58

### AA=83.06

### GA=68.18

A nonparametric test (Kruskal-Wallis) was performed on the following data comparing the mean of phenotypic data with genotype.

Rs2214653 (GRM3) with negative symptoms (p=0.046)

### GG=65.26

### AA=84.37

### GA=61.96

A nonparametric test (Kruskal-Wallis) was performed on the following data comparing the mean of phenotypic data with genotype.

Rs165774 (COMT) with suicide attempts (p=0.042). A Pearson chi-squared test was performed.

### [3] Assessment of Comorbidity and Health Problems (Mental and Physical)

Rs1800497 (ANK1) with TMTB (p=0.020)

CC=59.65 (mean)

### TT=61.11

### TC=60.32

A nonparametric test (Kruskal-Wallis) was performed on the following data comparing the mean of phenotypic data with genotype.

Rs4975646 (DAT) with GHQT (p=0.042)

### GG=74.42

### AA=102.57

### GA=71.31

A nonparametric test (Kruskal-Wallis) was performed on the following data comparing the mean of phenotypic data with genotype.

Rs4975646 (DAT) with GHQ3 (p=0.007). The AA genotype is associated with higher scores compared to the GG (0.007 Tukey and Bonferroni) and GA (0.008 Tukey and 0.009 Bonferroni) genotypes.

With GHQ4 (p=0.033). The AA genotype is associated with higher scores compared to the GA (0.052 Tukey) genotype.

With GHQT (p=0.019). The AA genotype is associated with higher scores compared to the GG (0.049 Tukey and 0.056 Bonferroni) and GA (0.014 Tukey and 0.015 Bonferroni) genotypes.

With TMTA (p=0.001). The AA genotype is associated with higher scores compared to the GG (0.001 Tukey and 0.002 Bonferroni) and GA (0.001 Tukey and Bonferroni) genotypes.

An ANOVA was performed on the following data, comparing the mean of phenotypic data with genotype.

Rs13161905 (DAT) with TMTA (p=0.013). The TT genotype is associated with higher scores compared to the CC (0.012 Tukey and 0.013 Bonferroni) and CT (0.021 Tukey and 0.024 Bonferroni) genotypes.

An ANOVA was performed on the following data, comparing the mean of phenotypic data with genotype.

Rs2214653 (GRM3) with TMTA (p=0.005). The AA genotype is associated with higher TMTA scores compared to the CC (0.019 Tukey and 0.021 Bonferroni) and CA (0.004 Tukey and 0.004 Bonferroni) genotypes.

With TMTB (p=0.043). The AA genotype is associated with higher TMTB scores compared to the CA (0.043 Tukey and 0.049 Bonferroni) genotype.

An ANOVA was performed on the following data, comparing the mean of phenotypic data with genotype.

Rs2870984 (PRODH) with GHQ1 (p=0.034). The AA genotype is associated with higher scores compared to the GG (0.041 Tukey and 0.047 Bonferroni) genotype.

With GHQT (p=0.047). The AA genotype is associated with higher scores compared to the GG (not significant 0.059 Tukey and 0.069 Bonferroni) genotype.

An ANOVA was performed on the following data, comparing the mean of phenotypic data with genotype.

Rs165774 (COMT) with impulsivity (p=0.006)

### GG=65.05

### AA=98.46

### GA=79.42

A nonparametric test (Kruskal-Wallis) was performed on the following data comparing the mean of phenotypic data with genotype.

Rs165774 (COMT) with impulsivity (p=0.006). The AA genotype is associated with higher scores compared to the GG (0.002 Tukey and Bonferroni) genotype.

An ANOVA was performed on the following data, comparing the mean of phenotypic data with genotype.

Rs10759 (RGS4) with impulsivity (p=0.050)

### CC=75.08

### AA=46.95

### CA=66.49

A nonparametric test (Kruskal-Wallis) was performed on the following data comparing the mean of phenotypic data with genotype.

Rs1997679 (DTNBP1) with AUDIT (p=0.028)

### CC=75.21

### TT=89.25

### CT=60.15

A nonparametric test (Kruskal-Wallis) was performed on the following data comparing the mean of phenotypic data with genotype.

Rs1997679 (DTNBP1) with AUDIT (p=0.011). The TT genotype is associated with higher scores compared to the CT (0.012 Tukey and 0.013 Bonferroni) genotype.

With TMTA (p=0.017). The TT genotype is associated with higher scores compared to the CC (0.013 Tukey and 0.014 Bonferroni) and CT (0.031 Tukey and 0.035 Bonferroni) genotypes.

An ANOVA was performed on the following data, comparing the mean of phenotypic data with genotype.

Rs4680 (COMT) with pulmonary function PF (p=0.039). Genotype AA has higher PF compared to GA (p=0.039 Tukey, 0.034 Bonferroni). An ANOVA was performed on the following data, comparing the mean of phenotypic data with genotype.

Rs9370822 (DTNBP1) with pulmonary function PF (p=0.0020). CC has higher PF compared to AA (p=0.002 Tukey and p=0.002 Bonferroni) and CA (p=0.006 Tukey and p=0.007 Bonferroni). An ANOVA was performed on the following data, comparing the mean of phenotypic data with genotype.

Rs3001371 (AKT1) with pulmonary function PF (p=0.040). Genotype CC has higher PF compared to TT and CT (not significant, p=0.108 and p=0.086 respectively with Tukey). An ANOVA was performed on the following data, comparing the mean of phenotypic data with genotype.

Rs4263535 (GABRA1) with cigs/day (p=0.018). Genotype GG has less cigs compared to genotypes AA (p=0.019 Tukey and p=0.021 Bonferroni). An ANOVA was performed on the following data, comparing the mean of phenotypic data with genotype.

Rs4263535 (GABRA1) with mgcigs/day (p=0.044). Genotype GG has less mg cigs/day compared to AA (0.059 Tukey and 0.069 Bonferroni). An ANOVA was performed on the following data, comparing the mean of phenotypic data with genotype.

Rs11133767 (DAT) with binge drinking (p=0.050). A Pearson chi-squared test was performed.

Rs9562919 (KPNA3) with g/hr alcohol (p=0.018). Genotype AA has higher g/hr alcohol compared to AT (p=0.018 Tukey and p=0.020 Bonferroni) and TT (p=0.076 Tukey and p=0.091 Bonferroni). An ANOVA was performed on the following data, comparing the mean of phenotypic data with genotype.

### [4] Medication Response (Adverse Medication Effects)

Rs13161905 (DAT) with glucose levels (p=0.010). The CT genotype has higher glucose level compared to the TT genotype (0.010 Tukey and Bonferroni). An ANOVA was performed on the following data, comparing the mean of phenotypic data with genotype.

Rs165774 (COMT) with prolactin levels (p=0.052). Genotype AA has higher prolactin compared to GG (0.040 Tukey HSD, 0.046 Bonferroni). An ANOVA was performed on the following data, comparing the mean of phenotypic data with genotype.

Rs9370822 (DTNBP1) with prolactin levels (p=0.034). CC has higher prolactin compared to AA (p=0.039 Tukey and p=0.045 Bonferroni). An ANOVA was performed on the following data, comparing the mean of phenotypic data with genotype.

Further SNP Correlations with Clinical and Phenotypic Schizophrenia Patient Data are shown in Tables 9 through 32.

TABLE 9

| | | rs6277L | ONSETAGE |
|---|---|---|---|
| rs6277L | Pearson Correlation | 1 | .183(*) |
| | Sig. (2-tailed) | | .027 |
| | N | 153 | 146 |
| ONSETAGE | Pearson Correlation | .183(*) | 1 |
| | Sig. (2-tailed) | .027 | |
| | N | 146 | 147 |

(*)Correlation is significant at the 0.05 level (2-tailed).

TABLE 10

| | | | RS40184Q | Family History | GHQT |
|---|---|---|---|---|---|
| Spearman's rho | RS40184Q | Correlation Coefficient | 1.000 | −.228(*) | .170(*) |
| | | Sig. (2-tailed) | — | .010 | .037 |
| | | N | 156 | 126 | 151 |
| | Family History | Correlation Coefficient | −.228(*) | 1.000 | .008 |
| | | Sig. (2-tailed) | .010 | — | .932 |
| | | N | 126 | 126 | 122 |
| | GHQT | Correlation Coefficient | .170(*) | .008 | 1.000 |
| | | Sig. (2-tailed) | .037 | .932 | — |
| | | N | 151 | 122 | 151 |

*Correlation is significant at the 0.05 level (2-tailed).

TABLE 11

| | | RS4975646Q | TMTA |
|---|---|---|---|
| RS4975646Q | Pearson Correlation | 1 | −.188(*) |
| | Sig. (2-tailed) | | .025 |
| | N | 156 | 142 |
| TMTA | Pearson Correlation | −.188(*) | 1 |
| | Sig. (2-tailed) | .025 | |
| | N | 142 | 142 |

(*)Correlation is significant at the 0.05 level (2-tailed).

TABLE 12

| | | | RS165774T | SUICIDE | DSMIV | IMPULSE |
|---|---|---|---|---|---|---|
| Spearman's | RS165774T | Correlation Coefficient | 1.000 | .172(*) | .171(*) | .257(**) |
| rho | | Sig. (2-tailed) | — | .048 | .034 | .002 |
| | | N | 154 | 132 | 154 | 145 |
| | SUICIDE | Correlation Coefficient | .172(*) | 1.000 | −.080 | −.046 |
| | ATTEMPTS | Sig. (2-tailed) | .048 | — | .357 | .600 |
| | | N | 132 | 134 | 134 | 131 |
| | DSMIV | Correlation Coefficient | .171(*) | −.080 | 1.000 | .092 |
| | | Sig. (2-tailed) | .034 | .357 | — | .268 |
| | | N | 154 | 134 | 156 | 147 |
| | IMPULSE | Correlation Coefficient | .257(**) | −.046 | .092 | 1.000 |
| | | Sig. (2-tailed) | .002 | .600 | .268 | — |
| | | N | 145 | 131 | 147 | 147 |

(*)Correlation is significant at the 0.05 level (2-tailed)
(**)Correlation is significant at the 0.01 level (2-tailed)

TABLE 13

| | | | RS4680T | Antipsychotic | IMPULSE |
|---|---|---|---|---|---|
| Spearman's rho | RS4680T | Correlation Coefficient | 1.000 | .161(*) | −.170(*) |
| | | Sig. (2-tailed) | — | .049 | .040 |
| | | N | 155 | 150 | 146 |
| | Antipsychotic | Correlation Coefficient | .161(*) | 1.000 | .116 |
| | | Sig. (2-tailed) | .049 | — | .168 |
| | | N | 150 | 151 | 142 |
| | IMPULSE | Correlation Coefficient | −.170(*) | .116 | 1.000 |
| | | Sig. (2-tailed) | .040 | .168 | — |
| | | N | 146 | 142 | 147 |

(*)Correlation is significant at the 0.05 level (2-tailed)

TABLE 14

| | | RS4263535U | NOADMISS | ONSETAGE | Mg/day cigs | CIG_DAY |
|---|---|---|---|---|---|---|
| RS4263535U | Pearson | 1 | −.133 | .205(*) | −.203(*) | −.202(*) |
| | Correlation | | | | | |
| | Sig. (2-tailed) | | .102 | .013 | .021 | .015 |
| | N | 156 | 152 | 147 | 130 | 146 |
| NOADMISS | Pearson | −.133 | 1 | −.191(*) | .378(**) | .362(**) |
| | Correlation | | | | | |
| | Sig. (2-tailed) | .102 | | .021 | .000 | .000 |
| | N | 152 | 152 | 146 | 129 | 144 |
| ONSETAGE | Pearson | .205(*) | −.191(*) | 1 | −.141 | −.141 |
| | Correlation | | | | | |
| | Sig. (2-tailed) | .013 | .021 | | .118 | .098 |
| | N | 147 | 146 | 147 | 124 | 140 |
| Mg/day cigs | Pearson | −.203(*) | .378(**) | −.141 | 1 | .864(**) |
| | Correlation | | | | | |
| | Sig. (2-tailed) | .021 | .000 | .118 | | .000 |
| | N | 130 | 129 | 124 | 130 | 130 |
| CIG_DAY | Pearson | −.202(*) | .362(**) | −.141 | .864(**) | 1 |
| | Correlation | | | | | |
| | Sig. (2-tailed) | .015 | .000 | .098 | .000 | |
| | N | 146 | 144 | 140 | 130 | 146 |

(*)Correlation is significant at the 0.05 level (2-tailed).
(**)Correlation is significant at the 0.01 level (2-tailed).

TABLE 15

| | | RS1997679W | LIFETIMEDRUGS | NOADMISS | TMTA |
|---|---|---|---|---|---|
| RS1997679W | Pearson Correlation | 1 | −.174(*) | −.160(*) | −.191(*) |
| | Sig. (2-tailed) | | .033 | .049 | .023 |
| | N | 156 | 150 | 152 | 142 |
| LIFETIMEDRUGS | Pearson Correlation | −.174(*) | 1 | .155 | .343(**) |
| | Sig. (2-tailed) | .033 | | .062 | .000 |
| | N | 150 | 150 | 146 | 142 |
| NOADMISS | Pearson Correlation | −.160(*) | .155 | 1 | .036 |
| | Sig. (2-tailed) | .049 | .062 | | .675 |
| | N | 152 | 146 | 152 | 139 |
| TMTA | Pearson Correlation | −.191(*) | .343(**) | .036 | 1 |
| | Sig. (2-tailed) | .023 | .000 | .675 | |
| | N | 142 | 142 | 139 | 142 |

(*)Correlation is significant at the 0.05 level (2-tailed).
(**)Correlation is significant at the 0.01 level (2-tailed).

TABLE 16

| | | | RS1997679W | LIFETIMEDRUGS | NOADMISS | TMTA |
|---|---|---|---|---|---|---|
| Spearman's | RS1997679W | Correlation Coefficient | 1.000 | −.165(*) | −.122 | −.061 |
| rho | | Sig. (2-tailed) | — | .044 | .134 | .468 |
| | | N | 156 | 150 | 152 | 142 |
| | LIFETIMEDRUGS | Correlation Coefficient | −.165(*) | 1.000 | .114 | .339(**) |
| | | Sig. (2-tailed) | .044 | — | .171 | .000 |
| | | N | 150 | 150 | 146 | 142 |
| | NOADMISS | Correlation Coefficient | −.122 | −.114 | 1.000 | .121 |
| | | Sig. (2-tailed) | .134 | .171 | — | .157 |
| | | N | 152 | 146 | 152 | 139 |
| | TMTA | Correlation Coefficient | −.061 | .339(**) | .121 | 1.000 |
| | | Sig. (2-tailed) | .468 | .000 | .157 | — |
| | | N | 142 | 142 | 139 | 142 |

(*)Correlation is significant at the 0.05 level (2-tailed).
(**)Correlation is significant at the 0.01 level (2-tailed).

TABLE 17

| | | | | PROLACTIN | |
|---|---|---|---|---|---|
| | | RS9370822W | NOADMISS | MCGS/L | PF |
| RS9370822W | Pearson Correlation | 1 | .194(*) | −.213(**) | −.256(**) |
| | Sig. (2-tailed) | | .017 | .010 | .002 |
| | N | 155 | 151 | 146 | 145 |
| NOADMISS | Pearson Correlation | .194(*) | 1 | −.131 | −.125 |
| | Sig. (2-tailed) | .017 | | .118 | .137 |
| | N | 151 | 152 | 144 | 143 |
| PROLACTIN MCGS/L | Pearson Correlation | −.213(**) | −.131 | 1 | −.056 |
| | Sig. (2-tailed) | .010 | .118 | | .510 |
| | N | 146 | 144 | 147 | 143 |
| PF | Pearson Correlation | −.256(**) | −.125 | −.056 | 1 |
| | Sig. (2-tailed) | .002 | .137 | .510 | |
| | N | 145 | 143 | 143 | 146 |

(*)Correlation is significant at the 0.05 level (2-tailed).
(**)Correlation is significant at the 0.01 level (2-tailed).

TABLE 18

| | | | | AUDITseverity |
|---|---|---|---|---|
| | | | RS2842030HH | of alcohol use |
| Spearman's rho | RS2842030HH | Correlation Coefficient | 1.000 | .178(*) |
| | | Sig. (2-tailed) | — | .039 |
| | | N | 152 | 136 |
| | AUDITseverity of | Correlation Coefficient | .178(*) | 1.000 |
| | alcohol use | Sig. (2-tailed) | .039 | — |
| | | N | 136 | 140 |

(*)Correlation is significant at the 0.05 level (2-tailed).

TABLE 19

| | | | RS3001371MM | UKUsexulsideeffect | PF |
|---|---|---|---|---|---|
| Spearman's | RS3001371MM | Correlation Coefficient | 1.000 | −.182(*) | .209(*) |
| rho | | Sig. (2-tailed) | — | .038 | .015 |
| | | N | 143 | 130 | 135 |
| | UKUsexualsideeffect | Correlation Coefficient | −.182(*) | 1.000 | −.107 |
| | | Sig. (2-tailed) | .038 | — | .209 |
| | | N | 130 | 143 | 140 |
| | PF | Correlation Coefficient | .209(*) | −.107 | 1.000 |
| | | Sig. (2-tailed) | .015 | .209 | — |
| | | N | 135 | 140 | 146 |

(*)Correlation is significant at the 0.05 level (2-tailed).

TABLE 20

| | | RS2238733NN | BINGE | GRAMS_H |
|---|---|---|---|---|
| RS2238733NN | Pearson Correlation | 1 | −.176(*) | .176(*) |
| | Sig. (2-tailed) | | .046 | .049 |
| | N | 141 | 129 | 126 |
| BINGE | Pearson Correlation | −.176(*) | 1 | −.516(**) |
| | Sig. (2-tailed) | .046 | | .000 |
| | N | 129 | 143 | 139 |
| GRAMS_H | Pearson Correlation | .176(*) | −.516(**) | 1 |
| | Sig. (2-tailed) | .049 | .000 | |
| | N | 126 | 139 | 140 |

(*)Correlation is significant at the 0.05 level (2-tailed).
(**)Correlation is significant at the 0.01 level (2-tailed).

TABLE 21

| | | | RS2238733NN | BINGE | GRAMS_H |
|---|---|---|---|---|---|
| Spearman's rho | RS2238733NN | Correlation Coefficient | 1.000 | −.185(*) | .171 |
| | | Sig. (2-tailed) | — | .035 | .056 |
| | | N | 141 | 129 | 126 |
| | BINGE | Correlation Coefficient | −.185(*) | 1.000 | −.794(**) |
| | | Sig. (2-tailed) | .035 | — | .000 |
| | | N | 129 | 143 | 139 |
| | GRAMS_H | Correlation Coefficient | .171 | −.794(**) | 1.000 |
| | | Sig. (2-tailed) | .056 | .000 | — |
| | | N | 126 | 139 | 140 |

(*)Correlation is significant at the 0.05 level (2-tailed).
(**)Correlation is significant at the 0.01 level (2-tailed).

TABLE 22

| | | | INOUT Pt | RS2870984NN |
|---|---|---|---|---|
| Spearman's rho | INOUT Pt | Correlation Coefficient | 1.000 | −.270(**) |
| | | Sig. (2-tailed) | . | .001 |
| | | N | 155 | 151 |
| | RS2870984NN | Correlation Coefficient | −.270(**) | 1.000 |
| | | Sig. (2-tailed) | .001 | . |
| | | N | 151 | 152 |

(**)Correlation is significant at the 0.01 level (2-tailed).

TABLE 23

| | | | | | PROLACTIN |
|---|---|---|---|---|---|
| | | | RS1800497AA | SUICIDE | MCGS/L |
| Spearman's | RS1800497AA | Correlation Coefficient | 1.000 | −.197(*) | −.288(**) |
| rho | | Sig. (2-tailed) | — | .029 | .001 |
| | | N | 144 | 123 | 135 |
| | SUICIDE | Correlation Coefficient | −.197(*) | 1.000 | .204(*) |
| | | Sig. (2-tailed) | .029 | — | .020 |
| | | N | 123 | 134 | 131 |
| | PROLACTIN MCGS/L | Correlation Coefficient | −.288(**) | .204(*) | 1.000 |
| | | Sig. (2-tailed) | .001 | .020 | — |
| | | N | 135 | 131 | 147 |

(*)Correlation is significant at the 0.05 level (2-tailed).
(**)Correlation is significant at the 0.01 level (2-tailed).

TABLE 24

| | | RS2214653FF | Family History | TMTA |
|---|---|---|---|---|
| RS2214653FF | Pearson Correlation | 1 | .215(*) | −.186(*) |
| | Sig. (2-tailed) | | .022 | .035 |
| | N | 142 | 113 | 128 |
| Family History | Pearson Correlation | .215(*) | 1 | −.257(**) |
| | Sig. (2-tailed) | .022 | | .005 |
| | N | 113 | 126 | 120 |
| TMTA | Pearson Correlation | −.186(*) | −.257(**) | 1 |
| | Sig. (2-tailed) | .035 | .005 | |
| | N | 128 | 120 | 142 |

(*)Correlation is significant at the 0.05 level (2-tailed).
(**)Correlation is significant at the 0.01 level (2-tailed).

TABLE 25

| | | | RS2214653FF | Family History | TMTA |
|---|---|---|---|---|---|
| Spearman's | RS2214653FF | Correlation Coefficient | 1.000 | .199(*) | −.086 |
| rho | | Sig. (2-tailed) | — | .035 | .332 |
| | | N | 142 | 113 | 128 |
| | Family History | Correlation Coefficient | .199(*) | 1.000 | −.290(**) |
| | | Sig. (2-tailed) | .035 | — | .001 |
| | | N | 113 | 126 | 120 |
| | TMTA | Correlation Coefficient | −.086 | −.290(**) | 1.000 |
| | | Sig. (2-tailed) | .332 | .001 | — |
| | | N | 128 | 120 | 142 |

(*)Correlation is significant at the 0.05 level (2-tailed).
(**)Correlation is significant at the 0.01 level (2-tailed).

TABLE 26

| | | | RS10759HH | IMPULSE |
|---|---|---|---|---|
| Spearman's | RS10759HH | Correlation | 1.000 | .189(*) |
| rho | | Coefficient | | |
| | | Sig. (2-tailed) | — | .027 |
| | | N | 145 | 137 |
| | IMPULSE | Correlation | .189(*) | 1.000 |
| | | Coefficient | | |
| | | Sig. (2-tailed) | .027 | — |
| | | N | 137 | 147 |

(*)Correlation is significant at the 0.05 level (2-tailed).

TABLE 27

| | | RS9562919LL | GRAMS_H | Family History |
|---|---|---|---|---|
| RS9562919LL | Pearson Correlation | 1 | .196(*) | −.193(*) |
| | Sig. (2-tailed) | | .023 | .034 |
| | N | 150 | 134 | 121 |
| GRAMS_H | Pearson Correlation | .196(*) | 1 | .079 |
| | Sig. (2-tailed) | .023 | | .390 |
| | N | 134 | 140 | 122 |
| Family History | Pearson Correlation | −.193(*) | .079 | 1 |
| | Sig. (2-tailed) | .034 | .390 | |
| | N | 121 | 122 | 126 |

(*)Correlation is significant at the 0.05 level (2-tailed).

TABLE 28

| | | | | | Family |
|---|---|---|---|---|---|
| | | | RS9562919LL | GRAMS_H | History |
| Spearman's | RS9562919LL | Correlation Coefficient | 1.000 | .138 | −.192(*) |
| rho | | Sig. (2-tailed) | — | .113 | .035 |
| | | N | 150 | 134 | 121 |
| | GRAMS_H | Correlation Coefficient | .138 | 1.000 | .009 |
| | | Sig. (2-tailed) | .113 | — | .917 |
| | | N | 134 | 140 | 122 |
| | Family History | Correlation Coefficient | −.192(*) | .009 | 1.000 |
| | | Sig. (2-tailed) | .035 | .917 | — |
| | | N | 121 | 122 | 126 |

(*)Correlation is significant at the 0.05 level (2-tailed).

TABLE 29

| | | RS2273816LL | LIFETIMEDRUGS | ATTENT | Mg/day cigs |
|---|---|---|---|---|---|
| RS2273816LL | Pearson Correlation | 1 | .209(*) | −.172(*) | −.105 |
| | Sig. (2-tailed) | | .013 | .042 | .248 |
| | N | 148 | 142 | 140 | 123 |
| LIFETIMEDRUGS | Pearson Correlation | .209(*) | 1 | .037 | −.150 |
| | Sig. (2-tailed) | .013 | | .658 | .088 |
| | N | 142 | 150 | 148 | 130 |
| ATTENT | Pearson Correlation | −.172(*) | .037 | 1 | −.026 |
| | Sig. (2-tailed) | .042 | .658 | | .769 |
| | N | 140 | 148 | 148 | 128 |
| Mg/day cigs | Pearson Correlation | −.105 | −.150 | −.026 | 1 |
| | Sig. (2-tailed) | .248 | .088 | .769 | |
| | N | 123 | 130 | 128 | 130 |

(*)Correlation is significant at the 0.05 level (2-tailed).

TABLE 30

| | | | | | | Mg/day |
|---|---|---|---|---|---|---|
| | | | RS2273816LL | LIFETIMEDRUGS | ATTENT | cigs |
| Spearman's | RS2273816LL | Correlation Coefficient | 1.000 | .202(*) | −.185(*) | −.198(*) |
| rho | | Sig. (2-tailed) | — | .016 | .028 | .028 |
| | | N | 148 | 142 | 140 | 123 |
| | LIFETIMEDRUGS | Correlation Coefficient | .202(*) | 1.000 | .010 | −.283(**) |
| | | Sig. (2-tailed) | .016 | — | .901 | .001 |
| | | N | 142 | 150 | 148 | 130 |
| | ATTENT | Correlation Coefficient | −.185(*) | .010 | 1.000 | .059 |
| | | Sig. (2-tailed) | .028 | .901 | — | .512 |
| | | N | 140 | 148 | 148 | 128 |
| | Mg/day cigs | Correlation | −.198(*) | −.283(**) | .059 | 1.000 |
| | | Coefficient | | | | |
| | | Sig. (2-tailed) | .028 | .001 | .512 | — |
| | | N | 123 | 130 | 128 | 130 |

(*)Correlation is significant at the 0.05 level (2-tailed).
(**)Correlation is significant at the 0.01 level (2-tailed).

TABLE 31

| | | RS2528856JJ | GLU |
|---|---|---|---|
| RS2528856JJ | Pearson Correlation | 1 | −.246(**) |
| | Sig. (2-tailed) | | .008 |
| | N | 143 | 117 |
| GLU | Pearson Correlation | −.246(**) | 1 |
| | Sig. (2-tailed) | .008 | |
| | N | 117 | 129 |

(**)Correlation is significant at the 0.01 level (2-tailed).

### Example 7

### SNP Correlations with Prolactin Levels of Schizophrenia Patients

### Adverse Drug Reaction Effects

TABLE 32

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 546.48 | 153 | 614.50 | 141 | 0.037 |
| Risperidone | 666.75 | 44 | 518.06 | 18 | 0.792 |
| Olanzapine | 395.85 | 40 | 529.00 | 26 | 0.046 |
| Clozapine | 532.23 | 43 | 551.87 | 47 | 0.618 |
| Typical | 637.83 | 29 | 723.90 | 29 | 0.034 |

Table 32 Serum prolactin levels in patients with allele 1 or allele 2 of the rs40184 (DA I) polymorphism receiving antipsychotic medication for schizophrenia

TABLE 33

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 538.06 | 212 | 710.45 | 78 | 0.000 |
| Risperidone | 668.15 | 40 | 572.80 | 20 | 0.850 |
| Olanzapine | 400.02 | 49 | 587.47 | 17 | 0.002 |
| Clozapine | 566.80 | 66 | 505.59 | 22 | 0.678 |
| Typical | 542.48 | 44 | 1115.79 | 14 | 0.000 |

Table 33 Serum prolactin levels in patients with allele 1 or allele 2 of the rs165774 (COMT) polymorphism receiving antipsychotic medication for schizophrenia.

TABLE 34

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 543.13 | 157 | 626.70 | 135 | 0.009 |
| Risperidone | 713.86 | 28 | 549.23 | 34 | 0.651 |
| Olanzapine | 308 | 33 | 614 | 31 | 0.000 |
| Clozapine | 613.00 | 54 | 436.72 | 36 | 0.096 |
| Typical | 534.76 | 33 | 873.80 | 25 | 0.006 |

Table 34 Serum prolactin levels in patients with allele 1 or allele 2 of the rs4680 (COMT) polymorphism receiving antipsychotic medication for schizophrenia.

TABLE 35

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 692.75 | 118 | 499.21 | 174 | 0.011 |
| Risperidone | 703.75 | 32 | 538.07 | 30 | 0.067 |
| Olanzapine | 669.11 | 18 | 365.50 | 48 | 0.005 |
| Clozapine | 562.93 | 41 | 512.68 | 47 | 0.611 |
| Typical | 853.09 | 23 | 567.69 | 35 | 0.478 |

Table 35 Serum prolactin levels in patients with allele 1 or allele 2 of the rs9370822 (DTNBP1) polymorphism receiving antipsychotic medication for schizophrenia.

TABLE 36

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 542.03 | 199 | 720.69 | 71 | 0.553 |
| Risperidone | 542.49 | 43 | 1179.18 | 11 | 0.002 |
| Olanzapine | 456.58 | 48 | 496.60 | 10 | 0.666 |
| Clozapine | 487.22 | 55 | 568.72 | 29 | 0.744 |
| Typical | 645.74 | 42 | 839.21 | 14 | 0.180 |

Table 36 Serum prolactin levels in patients with allele 1 or allele 2 of the rs1800497 (ANKK1) polymorphism receiving antipsychotic medication for schizophrenia.

TABLE 37

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 601.23 | 211 | 522.86 | 83 | 0.767 |
| Risperidone | 662.04 | 45 | 521.76 | 17 | 0.541 |
| Olanzapine | 507.61 | 41 | 351.04 | 25 | 0.044 |
| Clozapine | 564.96 | 70 | 463.85 | 20 | 0.523 |
| Typical | 676.75 | 44 | 693.79 | 14 | 0.333 |

Table 37 Serum prolactin levels in patients with allele 1 or allele 2 of the rs1997679 (DTNBP1) polymorphism receiving antipsychotic medication for schizophrenia.

TABLE 38

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 580.87 | 156 | 579.13 | 110 | 0.726 |
| Risperidone | 751.79 | 29 | 545.31 | 29 | 0.088 |
| Olanzapine | 404.69 | 42 | 596.06 | 16 | 0.002 |
| Clozapine | 468.82 | 39 | 519.81 | 43 | 0.397 |
| Typical | 697.25 | 36 | 707.44 | 16 | 0.498 |

Table 38 Serum prolactin levels in patients with allele 1 or allele 2 of the rs2214653 (GRM3) polymorphism receiving antipsychotic medication for schizophrenia

TABLE 39

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 681.87 | 55 | 595.11 | 150 | 0.069 |
| Risperidone | 1005.57 | 14 | 662.36 | 28 | 0.087 |
| Olanzapine | 368.36 | 14 | 427.33 | 30 | 0.965 |
| Clozapine | 746.06 | 18 | 441 | 44 | 0.001 |
| Typical | 537.67 | 9 | 794.37 | 38 | 0.403 |

Table 39 Serum prolactin levels in patients with allele 1 or allele 2 of the rs2770297 (HTR2A) polymorphism receiving antipsychotic medication for schizophrenia.

TABLE 40

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 565.32 | 270 | 771.64 | 22 | 0.362 |
| Risperidone | 648.69 | 58 | 197 | 2 | 0.223 |
| Olanzapine | 442.06 | 62 | 545 | 4 | 0.417 |
| Clozapine | 471.60 | 80 | 1109.60 | 10 | 0.000 |
| Typical | 680.86 | 58 | — | 0 | — |

Table 40 Serum prolactin levels in patients with allele 1 or allele 2 of the rs2870984 (PRODH) polymorphism receiving antipsychotic medication for schizophrenia.

TABLE 41

| | Allele 1 | | Allele 2 | | |
|---|---|---|---|---|---|
| Antipsychotic | Prolactin level | | Prolactin level | | |
| group | MCGS/L mean | n | MCGS/L mean | n | P |
| All antipsychotics | 573.90 | 269 | 646.16 | 19 | 0.343 |
| Risperidone | 659.04 | 56 | 278 | 4 | 0.146 |
| Olanzapine | 457.56 | 62 | 304.75 | 4 | 0.170 |
| Clozapine | 530.77 | 81 | 575 | 5 | 0.799 |
| Typical | 614 | 56 | 2550.00 | 2 | 0.009 |

Table 41 Serum prolactin levels in patients with allele 1 or allele 2 of the rs5747933 (PRODH) polymorphism receiving antipsychotic medication for schizophrenia.

### Example 8

### Association Between Polymorphism and Positive and Negative Symptoms Scale Total (PANSST)

Tables 42 to 50 provide data on drug responses of Risperidone, Olanzapine and Clozapine together with all antipsychotics and typical groups. The data are correlated into drug groups with a significant association between the polymorphism on allele 1 and/or allele 2 and the Positive and Negative Symptoms Scale Total (PANSST) being significant at P<0.05. Significant correlations are shown in bold.

TABLE 42

| Antipsychotic | Allele 1 | | Allele 2 | | |
|---|---|---|---|---|---|
| group | PANSST mean | n | PANSST mean | n | P |
| All antipsychotics | 45.68 | 203 | 44.49 | 103 | 0.110 |
| Risperidone | 45.32 | 41 | 45.46 | 26 | 0.182 |
| Olanzapine | 44.07 | 55 | 42.93 | 15 | 0.865 |
| Clozapine | 46.77 | 60 | 44.06 | 35 | 0.035 |
| Typical | 47.43 | 35 | 46.53 | 19 | 0.313 |

Table 42 PANSST levels in patients with allele 1 or allele 2 of the rs13161905 (DAT) polymorphism receiving antipsychotic medication for schizophrenia.

TALE 43

| Antipsychotic | Allele 1 | | Allele 2 | | |
|---|---|---|---|---|---|
| group | PANSST mean | n | PANSST mean | n | P |
| All antipsychotics | 45.23 | 277 | 46.26 | 23 | 0.006 |
| Risperidone | 45.40 | 60 | 39 | 2 | 0.210 |
| Olanzapine | 44.15 | 66 | 38.50 | 4 | 0.058 |
| Clozapine | 45.26 | 81 | 50.00 | 11 | 0.015 |
| Typical | 47.36 | 56 | | 0 | na |

Table 43 PANSST levels in patients with allele I or allele 2 of the rs2870984 (PRODB) polymorphism receiving antipsychotic medication for schizophrenia.

TABLE 44

| Antipsychotic | Allele 1 | | Allele 2 | | |
|---|---|---|---|---|---|
| group | PANSST mean | n | PANSST mean | n | P |
| All antipsychotics | 43.72 | 163 | 46.20 | 139 | 0.108 |
| Risperidone | 43.47 | 38 | 45.84 | 25 | 0.502 |
| Olanzapine | 43.12 | 42 | 44.89 | 28 | 0.515 |
| Clozapine | 45.39 | 46 | 44.98 | 47 | 0.847 |
| Typical | 50.73 | 15 | 46.12 | 41 | 0.002 |

Table 44 PANSST levels in patients with allele 1 or allele 2 of the rs6277 (DRD2) polymorphism receiving antipsychotic medication for schizophrenia.

TABLE 45

| Antipsychotic | Allele 1 | | Allele 2 | | |
|---|---|---|---|---|---|
| group | PANSST mean | n | PANSST mean | n | P |
| All antipsychotics | 45.90 | 106 | 45.04 | 202 | 0.014 |
| Risperidone | 44.50 | 20 | 45.74 | 47 | 0.980 |
| Olanzapine | 43.66 | 29 | 43.95 | 41 | 0.443 |
| Clozapine | 47.65 | 34 | 44.72 | 61 | 0.096 |
| Typical | 50.73 | 15 | 46.12 | 41 | 0.027 |

Table 45 PANSST levels in patients with allele 1 or allele 2 of the rs11133767 (DAD) polymorphism receiving antipsychotic medication for schizophrenia.

TABLE 46

| Antipsychotic | Allele 1 | | Allele 2 | | |
|---|---|---|---|---|---|
| group | PANSST mean | n | PANSST mean | n | P |
| All antipsychotics | 45.48 | 160 | 45.19 | 148 | 0.011 |
| Risperidone | 46 | 35 | 44.69 | 32 | 0.078 |
| Olanzapine | 43.20 | 42 | 44.46 | 28 | 0.471 |
| Clozapine | 44.61 | 44 | 46.76 | 51 | 0.333 |
| Typical | 50.97 | 29 | 43.48 | 27 | 0.019 |

Table 46 PANSST levels in patients with allele 1 or allele 2 of the rs40184 (DAT) polymorphism receiving antipsychotic medication for schizophrenia.

TABLE 47

| Antipsychotic | Allele 1 | | Allele 2 | | |
|---|---|---|---|---|---|
| group | PANSST mean | n | PANSST mean | n | P |
| All antipsychotics | 43.24 | 63 | 45.88 | 245 | 0.203 |
| Risperidone | 40.05 | 19 | 47.48 | 48 | 0.134 |
| Olanzapine | 41.71 | 7 | 44.06 | 63 | 0.088 |
| Clozapine | 44.43 | 21 | 46.15 | 74 | 0.818 |
| Typical | 46.10 | 10 | 47.63 | 46 | 0.020 |

Table 47 PANSST levels in patients with allele 1 or allele 2 of the rs4263535 (GABRA1) polymorphism receiving antipsychotic medication for schizophrenia.

TABLE 48

| Antipsychotic | Allele 1 | | Allele 2 | | |
|---|---|---|---|---|---|
| group | PANSST mean | n | PANSST mean | n | P |
| All antipsychotics | 43.60 | 60 | 45.63 | 158 | 0.869 |
| Risperidone | 43.88 | 16 | 45.67 | 30 | 0.099 |
| Olanzapine | 41.20 | 15 | 46.69 | 32 | 0.076 |
| Clozapine | 47.05 | 20 | 43.39 | 46 | 0.460 |
| Typical | 39.44 | 9 | 48.00 | 38 | 0.001 |

Table 48 PANSST levels in patients with allele 1 or allele 2 of the rs2770297 (HTR2A) polymorphism receiving antipsychotic medication for schizophrenia.

TABLE 49

| Antipsychotic | Allele 1 | | Allele 2 | | |
|---|---|---|---|---|---|
| group | PANSST mean | n | PANSST mean | n | P |
| All antipsychotics | 45.49 | 211 | 45.18 | 73 | 0.145 |
| Risperidone | 46.23 | 48 | 41 | 11 | 0.394 |
| Olanzapine | 44.57 | 51 | 41.36 | 11 | 0.267 |
| Clozapine | 44.47 | 58 | 47.58 | 31 | 0.100 |
| Typical | 48.56 | 41 | 45.92 | 13 | 0.020 |

Table 49 PANSST levels in patients with allele 1 or allele 2 of the rs1800497 (PRODH) polymorphism receiving antipsychotic medication for schizophrenia.

TABLE 50

| Antipsychotic | Allele 1 | | Allele 2 | | |
|---|---|---|---|---|---|
| group | PANSST mean | n | PANSST mean | n | P |
| All antipsychotics | 45.46 | 151 | 45.18 | 145 | 0.798 |
| Risperidone | 46.35 | 34 | 43.81 | 31 | 0.840 |
| Olanzapine | 41.24 | 34 | 45.41 | 34 | 0.378 |
| Clozapine | 45.58 | 45 | 46.24 | 46 | 0.122 |
| Typical | 50.97 | 29 | 43.84 | 25 | 0.017 |

Table 50 PANSST levels in patients with allele 1 or allele 2 of the Rs9562919 (KPNA3) polymorphism receiving antipsychotic medication for schizophrenia.

### Example 9

### Clinical Associations with the C957T DRD2 Polymorphism

### Rs 6277

The following associations were identified.

### Clinical History

The CT and CC genotypes are associated with older onset age (p=0.023).

### Comorbidities

The T allele is associated with higher levels of grams of alcohol use per hour (p=0.014).

### Adverse Effects

(i) The T allele is associated with a higher AIMS total score (abnormal involuntary movement scale, total score out of 40) [p=0.010].

(ii) The T allele is associated with poorer attention score (PANSS Rating Scale—General Scale GII: poor attention) [p=0.001].

### Clinical Response

(i) The C allele is associated with higher CPZE/kg (Chlorpromazine equivalent in mg/kg body weight for primary antipsychotic drug) scores (p=0.041).

(ii) The T allele is associated with more significant Negative Symptoms score (PANSS Rating Scale-negative scale-Likert scoring system) [p=0.012].

(iii) The C allele is associated with higher CPZE (Chlorpromazine equivalent in mg for primary antipsychotic drug) scores (p=0.018).

### Example 10

### Adverse Drug Reaction Effects with the C957T DRD2 Polymorphism

### Rs 6277

### Olazapine

(i) When patients were prescribed the antipsychotic drug Olazapine, the C allele was associated with a higher Barnes total score (Barnes Akathisia Scale Total Score out of 14) [p=0.015], indicating greater motor restlessness causing distress.

(ii) When patients were prescribed the antipsychotic drug Olazapine, the T allele was associated with a higher poor attention score (PANSS Rating Scale-General Scale GII: poor attention) [p=0.014].

(iii) When patients were prescribed the antipsychotic drug Olazapine, the T allele was associated with a higher TMTB score (Trail Making Test (Part B) time in seconds) [p=0.017], indicating poorer global cognitive functioning, particularly cognitive flexibility.

### Clozapine

(i) When patients were prescribed the antipsychotic drug Clozapine, the T allele was associated with poorer attention score (PANSS Rating Scale-General Scale GII: poor attention) [p=0.037].

(ii) When patients were prescribed the antipsychotic drug Clozapine,

the T allele was associated with a higher Barnes total score (Barnes Akathisia Scale Total Score out of 14) [p=0.002], indicating greater motor restlessness causing distress.

### Example 11

### Clinical Associations with the Taq 1A Polymorphism

### Rs 1800497

### Adverse Effects

(i) The TT or A1A1 genotype is associated with a higher Barnes total score (Barnes Akathisia Scale Total Score out of 14) [p=0.040], indicating greater motor restlessness causing distress.

(ii) The TT or A1A1 genotype is associated with increased levels of the hormone prolactin (mcgs/L) [p=0.027].

(iii) The T or A1 allele is associated with a higher TMTA (Trail Making Test [Part A] time in seconds) score (p=0.008), indicating poorer global cognitive functioning.

### Clinical Response

(i) The TT or A1A1 genotype is associated with increased CPZE (Chlorpromazine equivalent in mg for primary antipsychotic drug) levels (p=0.012).

(ii) The TT or A1A1 genotype is associated with increased CPZE/kg (Chlorpromazine equivalent in mg/kg body weight for primary antipsychotic drug) levels (p=0.004).

(iii) The TT or A1A1 genotype is associated with increased CPZE total/kg (Chlorpromazine equivalent in mg/kg body weight for total antipsychotic drug) levels (p=0.013).

### Clinical History

The T or A1 allele is associated with more hospital admissions (p=0.000).

### Example 12

### Adverse Drug Reaction Effects with the Taq 1A Polymorphism

### Rs 1800497

### Clozapine

### Adverse Effects

When patients were prescribed the antipsychotic drug Clozapine, the T or A1 allele was associated with a higher TMTA (Trail Making Test [Part A] time in seconds) score (p=0.000), indicating poorer global cognitive functioning.

### Clinical Response

When patients were prescribed the antipsychotic drug Clozapine, the T or A1 allele was associated with more significant positive symptoms (PANSS Rating Scale-positive scale-Likert scoring system) score (p=0.015).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Abi-Dargham et al, Proc. Natl. Acad. Sci. 97:8104-8109, 2000
Bassett et al, Biol Psychiatry, 46:882-891, 1999
Berrettini, Am J Med Genet, 123C:59-64, 2003
Bray et al, Am J Hum Genet, 73(1):152-161, 2003
Brennan, A M J Pharmacogenomics. 1(4):295-302, 2001
Burd et al, J Trauma 60(4):792-801, 2006
Callicott et al, Am. J. Psychiatry. 160:709-719, 2003
Cariello, Am. J. Human Genetics 42:726-734, 1988
Carter et al, BMC Bioinformatics 7(1):60, 2006
Chowdari et al, Hum Mol Genet, 11(12):1373-1380, 2002
Conner et al, Proc. Natl. Acad. Sci. USA 80:278-282, 1983
Cotton et al, Proc. Natl. Acad. Sci. USA 87:4033-40371988
Donders et al, J Clin Epidemiol 59(10):1087-1091, 2006
Duan et al, Hum Mol Genet. 12:205-16, 2003
Dubretret et al, Schizophrenia Res, 49:202-212, 2001
Dubretret et al, Schizophrenia Res, 67:75-85, 2004
Edwards et al, Methods Mol. Biol. 226:287-94, 2003
Egan et al, Proc. Natl. Acad. Sci., USA, 98(12):6917-6922, 2001
Elghanian et al, Science 277:1078-1081, 1997
Finkelstein et al, Genomics 7:167-172, 1990
Fujii et al, Psychiatr Genet, 13:71-76, 2003
Gogos et al, Nat Genet, 21:434-439
Gottesman I I et al, Proc. Natl. Acad. Sci. USA. 58:199, 1967
Grompe et al, Proc. Natl. Acad. Sci. USA 86:5855-5892, 1989
Group, Am J Med Genet, 67:580-594, 1996
Hoenicka et al, Acta Psychiatr Scand, 114:435-438, 2006
Hanninen et al, Neuros Lett, 407:195-198, 2006
Kelly et al, Ir. J. Med. Sci. 172:37-40, 2003
Kinszler et al, Science 251:1366-1370, 1991
Kwok et al, Curr Issues Mol Biol 5(2):43-60, 2003
Lawford et al, Schizo Res, 73:31-37, 2005
Lewis et al, Am J Hum Genet, 73:34-48, 2003
Liu et al, Proc. Natl. Acad. USA, 99:3717-3722, 2002
MacDonald et al, Arch. Gen. Psychiatry. 60:57-65, 2003
Marti et al, Am J Med Genet, 144:46-50, 2002
McGue et al, Arch Gen Psych. 46:478-480
Mimics et al, Mol Psychiatry, 6:293-301, 2001
Millar et al, Science, 310:1187-1191, 2005
Miyamoto et al, Mol. Intervent. 3:27-39, 2004
Modrich, Ann. Rev. Genet. 25:229-253, 1991
Mowry et al, Mol. Psychiatry. 2004
Murphy, Lancet, 359:426-430, 2002
Ni et al, J Neurosci, 19(10):3674-3680, 1999
Niendam et al, Am. J. Psychiatry. 160:2060-2062, 2003
Norton et al, Ann Med, 37(2):121-129, 2005
Norton et al, Curr Opin Psychiatr, 19:158-164, 2006
Novack et al, Proc. Natl. Acad. Sci. USA 83:586-590, 1986
Orita, et al, Proc. Natl. Acad. Sci. USA. 86:2766-2770, 1989
Owen et al, Mol. Psychiatry. 9:14-27, 2004
Owen et al, Psychiatric genetics and genomics: 247-266, 2002
Risch, Gen Epidemiol, 7:3-16, 1990
Ruano et al, Nucl. Acids Res. 17:8392, 1989
Scherer et al, Genomics, 31:230-233, 1996
Seeman et al, Proc. Natl. Acad. Sci. USA 97:7673-7675, 2000
Sheffield et al, Proc. Natl. Acad. Sci. USA 86:232-236, 1989
Shenk et al, Proc. Natl. Acad. Sci. USA 72:989-993, 1975
Shifman et al, Am J Hum Genet, 71:1296-1303, 2002
Talbot et al, J Clin Invest, 113:1353-1363, 2004
Taylor et al, Biotechniques 30:661-669, 2001
Tepest et al, Biol, Psychiatry. 54:1234-1240, 2003
Twyman et al, Pharmacogenomics. 4(1):67-79, 2003
van der Heijden et al, J Clin Epidemiol 59(10):1102-1109, 2006
Wartell et al, Nucl. Acids Res. 18:2699-2705, 1990
Wei et al, Neurosci Res, 52:342-346, 2005
Weickert et al, Arch Gen Psychiatry, 61:544-555, 2004
White et al, Genomics 12:301-306, 1992
Williams et al, Arch Gen Psychiatry, 61:336-344, 2004
Winterer et al, Arch. Gen. Psychiatry. 60:1158-1167, 2003
Yamada et al, Biol Psychiatry, 56:683-690, 2004

What is claimed is:

1. A method for identifying a genetic profile associated with schizophrenia or a related condition in an individual or a group of individuals, said method comprising screening individuals for a polymorphism including a mutation in a gene selected from the list in Table 2 which has a statistically significant linkage or association to symptoms or behavior characterizing schizophrenia or the related condition.

2. The method of claim 1 wherein the polymorphism is a single nucleotide polymorphism (SNP).

3. The method of claim 1 wherein the polymorphism is selected from a multi-nucleotide polymorphism (MNP) or nucleotide addition, insertion, substitution, deletion, rearrangement or microsatellite.

4. The method of claim 1 wherein the polymorphism is detected in a panel of two or more genes listed in Table 3.

5. The method of claim 4 wherein the polymorphism is detected in all the genes in Table 3.

6. The method of claim 1 wherein the condition related to schizophrenia is post traumatic stress disorder (PTSD) or an addiction selected from the list consisting of alcohol dependence, nicotine dependence and opioid dependence.

7. The method of claim 6 wherein the condition is schizophrenia.

8. A panel of genetic mutations providing a genetic marker set for schizophrenia or a related condition in an individual said genetic marker comprising from about one to about 100 polymorphisms in one or more genes listed in Table 2 wherein the presence of the polymorphisms is indicative of or a predisposition to developing schizophrenia or the related condition.

9. The panel of genetic mutations of claim 8 wherein the polymorphisms are in two or more genes in Table 3.

10. The panel of genetic mutations of claim 9 wherein the condition related to schizophrenia is PTSD or an addiction selected from the list consisting of alcohol dependence, nicotine dependence and opioid dependence.

11. The panel of genetic mutations of claim 10 wherein the condition is schizophrenia.

12. A method for identifying a genetic profile consistent with schizophrenia or a related condition in a individual, said method comprising obtaining or extracting a DNA sample from cells of said individual and screening for or otherwise detecting the presence from about one to about 100 polymorphisms in one or more genes listed in Table 2 having a statistical significant association with schizophrenia or the related condition wherein the presence of that genetic profile is indicative of schizophrenia or the related condition or that the individual is at risk of developing same.

13. The method of claim 12 wherein the polymorphism is a SNP.

14. The method of claim 12 wherein the polymorphism is selected from a MNP or a nucleotide addition, insertion, substitution, deletion, rearrangement or microsatellite.

15. The method of claim 12 wherein the polymorphisms are detected in two or more genes listed in Table 3.

16. The method of claim 12 wherein the condition related to schizophrenia is PTSD or an addiction selected from the list consisting of alcohol dependence, nicotine dependence and opioid dependence.

17. The method of claim 16 wherein the condition is schizophrenia.

18. A method for identifying a genetic basis behind diagnosing or treating schizophrenia or a related condition, said method comprising obtaining or extracting a DNA sample from cells of said individual and screening for or otherwise detecting the presence of from about one to about 100 polymorphisms in one or more genes listed in Table 2 with a statistical significant association with schizophrenia or a related condition wherein the presence of that genetic profile is indicative of schizophrenia or the related condition or that the individual is at risk of developing same or that the individual will respond to a particular treatment.

19. The method of claim 20 wherein the condition related to schizophrenia is PTSD or an addiction selected from the list consisting of alcohol dependence, nicotine dependence and opioid dependence.

20. The method of claim 19 wherein the condition is schizophrenia.

21. A method a method for determining the likelihood of a subject responding favorably to a particular drug in the treatment of schizophrenia or a related condition said method comprising obtaining or extracting a DNA sample from cells of said individual and screening for or otherwise detecting the presence of from about one to about 100 polymorphisms in one or more genes listed in Table 2 with a statistical significant association with schizophrenia or the related condition the presence of the SNP profile is indicative of the likelihood of the drug being effective.

22. The method of claim 23 wherein the condition related to schizophrenia is PTSD or an addiction selected from the list consisting of alcohol dependence, nicotine dependence and opioid dependence.

23. The method of claim 24 wherein the condition is schizophrenia.

24. A method for identifying a genetic basis behind diagnosing or treating schizophrenia or a related condition in an individual, said method comprising obtaining a biological sample from said individual and detecting a protein encoded by a nucleotide sequence having from about one to about 100 polymorphisms in one or more genes listed in Table 2 with a statistical significant association with schizophrenia or a related condition resulting in from about one to about 100 amino acid insertions, substitutions or deletions wherein the presence of an altered amino acid sequence is indicative of the presence of a polymorphism and the likelihood of schizophrenia or a related condition.

25. The method of claim 26 wherein the amino acid alteration is detected by a specific antibody which discriminates between the presence or absence of the amino acid alteration.

26. The method of claim 26 wherein the amino acid alteration is detected by amino acid sequencing.

27. The method of claim 26 wherein the amino acid alteration is detected by a change in protein activity or cell phenotype.

28. The method of claim 26 wherein the amino acid alteration is detected via the presence of a metabolite if the protein is associated with a biochemical pathway.


SEQUENCE LISTING (112 sequences)

SEQ ID NO: 1 - DNA, 30 bp, Homo sapiens
acgttggatgagccaccaccagctgactct

SEQ ID NO: 2 - DNA, 30 bp, Homo sapiens
acgttggatgattcttctctggtttggcgg

SEQ ID NO: 3 - DNA, 30 bp, Homo sapiens
acgttggatgagctgactcacacagtgatg

SEQ ID NO: 4 - DNA, 30 bp, Homo sapiens
acgttggatgcggttttgaaaggaactgcc

SEQ ID NO: 5 - DNA, 30 bp, Homo sapiens
acgttggatgatacttcgaagccactctgg

SEQ ID NO: 6 - DNA, 30 bp, Homo sapiens
acgttggatggcctaaatacctcaatcctg

SEQ ID NO: 7 - DNA, 30 bp, Homo sapiens
acgttggatgtgtaagaaagtagcagcccc

SEQ ID NO: 8 - DNA, 28 bp, Homo sapiens
acgttggatgctggattcattcttgtcc

SEQ ID NO: 9 - DNA, 30 bp, Homo sapiens
acgttggatgcacagtctcgcggcttttta

SEQ ID NO: 10 - DNA, 30 bp, Homo sapiens
acgttggatgaacacacccttgacaggtgc

SEQ ID NO: 11 - DNA, 30 bp, Homo sapiens
acgttggatgtgacaggtgcagtgagagag

SEQ ID NO: 12 - DNA, 30 bp, Homo sapiens
acgttggatgtcagtgctgcaggcacactt

SEQ ID NO: 13 - DNA, 29 bp, Homo sapiens
acgttggatgactgccatcatgcagcagg

SEQ ID NO: 14 - DNA, 30 bp, Homo sapiens
acgttggatgatcctcccttgatcagggtc

SEQ ID NO: 15 - DNA, 30 bp, Homo sapiens
acgttggatgtgaacgctgaacgtgccttc

SEQ ID NO: 16 - DNA, 30 bp, Homo sapiens
acgttggatgtgaagatgctgcctgctctg

SEQ ID NO: 17 - DNA, 30 bp, Homo sapiens
acgttggatgttttccaggtctgacaacgg

SEQ ID NO: 18 - DNA, 30 bp, Homo sapiens
acgttggatgacccagcggatggtggattt

SEQ ID NO: 19 - DNA, 30 bp, Homo sapiens
acgttggatgttccaatccgtgtccagggt

SEQ ID NO: 20 - DNA, 30 bp, Homo sapiens
acgttggatggaaactggacactgctgtta

SEQ ID NO: 21 - DNA, 30 bp, Homo sapiens
acgttggatgattggcagtcttgatggctc

SEQ ID NO: 22 - DNA, 30 bp, Homo sapiens
acgttggatgtagggagcccatgcaaatag

SEQ ID NO: 23 - DNA, 31 bp, Homo sapiens
acgttggatgccgtacaaaaatacaagagtg

SEQ ID NO: 24 - DNA, 30 bp, Homo sapiens
acgttggatgtaactctttgaggacagagc

SEQ ID NO: 25 - DNA, 30 bp, Homo sapiens
acgttggatgcgaaccatatgacttttgtg

SEQ ID NO: 26 - DNA, 30 bp, Homo sapiens
acgttggatggaagttttgctactagtgag

SEQ ID NO: 27 - DNA, 30 bp, Homo sapiens
acgttggatggccagtttttatctccagcc

SEQ ID NO: 28 - DNA, 30 bp, Homo sapiens
acgttggatggtttggaaactggcccagtc

SEQ ID NO: 29 - DNA, 29 bp, Homo sapiens
acgttggatgcataactaattgtagtcca

SEQ ID NO: 30 - DNA, 30 bp, Homo sapiens
acgttggatggagtttcaggctagcttctc

SEQ ID NO: 31 - DNA, 29 bp, Homo sapiens
acgttggatgacctgcagcaccgccacct

SEQ ID NO: 32 - DNA, 30 bp, Homo sapiens
acgttggatgttcaccaaatggctgtggag

SEQ ID NO: 33 - DNA, 30 bp, Homo sapiens
acgttggatggaaagcacaaagcagctgag

SEQ ID NO: 34 - DNA, 30 bp, Homo sapiens
acgttggatgtgctccagctctgggaagtg

SEQ ID NO: 35 - DNA, 30 bp, Homo sapiens
acgttggatgcagagatcggctatgaggac

SEQ ID NO: 36 - DNA, 30 bp, Homo sapiens
acgttggatgacctgtggtacatggcgttg

SEQ ID NO: 37 - DNA, 30 bp, Homo sapiens
acgttggatgcaacacagccatcctcaaag

SEQ ID NO: 38 - DNA, 30 bp, Homo sapiens
acgttggatgtgtgcagctcactccatcct

SEQ ID NO: 39 - DNA, 30 bp, Homo sapiens
acgttggatgacaacgtgctgtaggaaacc

SEQ ID NO: 40 - DNA, 30 bp, Homo sapiens
acgttggatggcccttctctctctgatgtt

SEQ ID NO: 41 - DNA, 30 bp, Homo sapiens
acgttggatgagtgtgatgaccataaaccc

SEQ ID NO: 42 - DNA, 30 bp, Homo sapiens
acgttggatgcctcaaatgtattcccaatg

SEQ ID NO: 43 - DNA, 29 bp, Homo sapiens
acgttggatgacaactgaaaaacacactc

SEQ ID NO: 44 - DNA, 30 bp, Homo sapiens
acgttggatgctcacagactgctgtcttac

SEQ ID NO: 45 - DNA, 17 bp, Homo sapiens
tgtctccacagcactcc

SEQ ID NO: 46 - DNA, 18 bp, Homo sapiens
tgtctccacagcactccc

SEQ ID NO: 47 - DNA, 18 bp, Homo sapiens
tgtctccacagcactcct

SEQ ID NO: 48 - DNA, 18 bp, Homo sapiens
tggggatgtaaaatggaa

SEQ ID NO: 49 - DNA, 19 bp, Homo sapiens
tggggatgtaaaatggaag

SEQ ID NO: 50 - DNA, 19 bp, Homo sapiens
tggggatgtaaaatggaat

SEQ ID NO: 51 - DNA, 23 bp, Homo sapiens
gacttttaccttttgagctgtta

SEQ ID NO: 52 - DNA, 24 bp, Homo sapiens
gacttttaccttttgagctgttac

SEQ ID NO: 53 - DNA, 24 bp, Homo sapiens
gacttttaccttttgagctgttat

SEQ ID NO: 54 - DNA, 26 bp, Homo sapiens
cccccacaccttgccaccaaataaag

SEQ ID NO: 55 - DNA, 27 bp, Homo sapiens
cccccacaccttgccaccaaataaagc

SEQ ID NO: 56 - DNA, 27 bp, Homo sapiens
cccccacaccttgccaccaaataaagt

SEQ ID NO: 57 - DNA, 26 bp, Homo sapiens
aaaatcaagtaatgattgatttgtag

SEQ ID NO: 58 - DNA, 27 bp, Homo sapiens
aaaatcaagtaatgattgatttgtaga

SEQ ID NO: 59 - DNA, 27 bp, Homo sapiens
aaaatcaagtaatgattgatttgtagg

SEQ ID NO: 60 - DNA, 16 bp, Homo sapiens
acatggcacctatgag

SEQ ID NO: 61 - DNA, 17 bp, Homo sapiens
acatggcacctatgagc

SEQ ID NO: 62 - DNA, 17 bp, Homo sapiens
acatggcacctatgagg

SEQ ID NO: 63 - DNA, 16 bp, Homo sapiens
gggcgcacatgggatg

SEQ ID NO: 64 - DNA, 17 bp, Homo sapiens
gggcgcacatgggatga

SEQ ID NO: 65 - DNA, 17 bp, Homo sapiens
gggcgcacatgggatgg

SEQ ID NO: 66 - DNA, 16 bp, Homo sapiens
ccttccttccactgcc

SEQ ID NO: 67 - DNA, 17 bp, Homo sapiens
ccttccttccactgccc

SEQ ID NO: 68 - DNA, 17 bp, Homo sapiens
ccttccttccactgcct

SEQ ID NO: 69 - DNA, 18 bp, Homo sapiens
ttcacaccttgtccttca

SEQ ID NO: 70 - DNA, 19 bp, Homo sapiens
ttcacaccttgtccttcac

SEQ ID NO: 71 - DNA, 19 bp, Homo sapiens
ttcacaccttgtccttcat

SEQ ID NO: 72 - DNA, 16 bp, Homo sapiens
cctcgtgctcctagtc

SEQ ID NO: 73 - DNA, 17 bp, Homo sapiens
cctcgtgctcctagtcc

SEQ ID NO: 74 - DNA, 17 bp, Homo sapiens
cctcgtgctcctagtct

SEQ ID NO: 75 - DNA, 23 bp, Homo sapiens
gggtgttcctgggtaaccctaga

SEQ ID NO: 76 - DNA, 24 bp, Homo sapiens
gggtgttcctgggtaaccctagac

SEQ ID NO: 77 - DNA, 24 bp, Homo sapiens
gggtgttcctgggtaaccctagat

SEQ ID NO: 78 - DNA, 20 bp, Homo sapiens
aaaatacaagagtgtcagga

SEQ ID NO: 79 - DNA, 21 bp, Homo sapiens
aaaatacaagagtgtcaggag

SEQ ID NO: 80 - DNA, 21 bp, Homo sapiens
aaaatacaagagtgtcaggat

SEQ ID NO: 81 - DNA, 24 bp, Homo sapiens
gacaatatgacttttgtgactgct

SEQ ID NO: 82 - DNA, 25 bp, Homo sapiens
gacaatatgacttttgtgactgcta

SEQ ID NO: 83 - DNA, 25 bp, Homo sapiens
gacaatatgacttttgtgactgctt

SEQ ID NO: 84 - DNA, 16 bp, Homo sapiens
ccccacccaaacccca

SEQ ID NO: 85 - DNA, 17 bp, Homo sapiens
ccccacccaaaccccac

SEQ ID NO: 86 - DNA, 17 bp, Homo sapiens
ccccacccaaaccccat

SEQ ID NO: 87 - DNA, 23 bp, Homo sapiens
actaattgtagtccaatttagac

SEQ ID NO: 88 - DNA, 24 bp, Homo sapiens
actaattgtagtccaatttagaca

SEQ ID NO: 89 - DNA, 24 bp, Homo sapiens
actaattgtagtccaatttagacg

SEQ ID NO: 90 - DNA, 16 bp, Homo sapiens
cgccacctccagcttg

SEQ ID NO: 91 - DNA, 17 bp, Homo sapiens
cgccacctccagcttgg

SEQ ID NO: 92 - DNA, 17 bp, Homo sapiens
cgccacctccagcttgt

SEQ ID NO: 93 - DNA, 17 bp, Homo sapiens
gtgaggacaggagggaa

SEQ ID NO: 94 - DNA, 18 bp, Homo sapiens
gtgaggacaggagggaac

SEQ ID NO: 95 - DNA, 18 bp, Homo sapiens
gtgaggacaggagggaaa

SEQ ID NO: 96 - DNA, 19 bp, Homo sapiens
caggaccccatcaacccca

SEQ ID NO: 97 - DNA, 20 bp, Homo sapiens
caggaccccatcaaccccac

SEQ ID NO: 98 - DNA, 20 bp, Homo sapiens
caggaccccatcaaccccat

SEQ ID NO: 99 - DNA, 26 bp, Homo sapiens
ccacagccatcctcaaagtgctggtc

SEQ ID NO: 100 - DNA, 27 bp, Homo sapiens
ccacagccatcctcaaagtgctggtca

SEQ ID NO: 101 - DNA, 27 bp, Homo sapiens
ccacagccatcctcaaagtgctggtcg

SEQ ID NO: 102 - DNA, 17 bp, Homo sapiens
tctggacggctaaagac

SEQ ID NO: 103 - DNA, 18 bp, Homo sapiens
tctggacggctaaagacc

SEQ ID NO: 104 - DNA, 18 bp, Homo sapiens
tctggacggctaaagact

SEQ ID NO: 105 - DNA, 21 bp, Homo sapiens
taccataaaccctgagcccca

SEQ ID NO: 106 - DNA, 22 bp, Homo sapiens
taccataaaccctgagccccac

SEQ ID NO: 107 - DNA, 22 bp, Homo sapiens
taccataaaccctgagccccat

SEQ ID NO: 108 - DNA, 27 bp, Homo sapiens
acataaaataatttacttctcattcag

SEQ ID NO: 109 - DNA, 28 bp, Homo sapiens
acataaaataatttacttctcattcagc

SEQ ID NO: 110 - DNA, 28 bp, Homo sapiens
acataaaataatttacttctcattcaga

SEQ ID NO: 111 - DNA, 20 bp, Homo sapiens
gaaagctgcatcaagagccc

SEQ ID NO: 112 - DNA, 21 bp, Homo sapiens
ttcactgtaaacccctggaga